(19) United States
(12) Patent Application Publication
(10) Pub. No.: US 2006/0155178 A1
(43) Pub. Date: Jul. 13, 2006

(54) Multi-dimensional elastic light scattering

(76) Inventors: Vadim Backman, Evanston, IL (US); Hemant Roy, Highland Park, IL (US); Ramesh Wali, Darien, IL (US); Young Kim, Skokie, IL (US); Yang Liu, Evanston, IL (US); Guillermo Ameer, Chicago, IL (US); Jian Yang, Evanston, IL (US); Antonio Webb, Chicago, IL (US); Josephine Allen, Evanston, IL (US)

Correspondence Address: EVAN LAW GROUP LLC, 566 WEST ADAMS, SUITE 350, CHICAGO, IL, 60661, US

(21) Appl. No.: 11/261,452

(22) Filed: Oct. 27, 2005

Related U.S. Application Data
(63) Continuation-in-part of application No. 10/945,354, filed on Sep. 20, 2004.
(60) Provisional application No. 60/556,642, filed on Mar. 26, 2004.
(60) Provisional application No. 60/622,673, filed on Oct. 27, 2004.

(51) Int. Cl. A61B 5/00

(52) U.S. Cl. 600/315

(57) ABSTRACT

A method of examining a sample includes measuring, as function of wavelength of light elastically scattered from the sample, at least 2 properties, selected from the group consisting of scattering angle theta of the light, scattering angle phi of the light, and polarization of the light. The scattering angle theta is an angle between backward direction and direction of propagation of the light, and scattering angle phi is an angle between incident light polarization and projection of direction of the light propagation onto a plane in which incident electric field oscillates.

### CROSS REFERENCE TO RELATED APPLICATION

This application is a Continuation-in-Part of U.S. patent application Ser. No. 10/945,354 filed 24 Mar. 2005, which claims the benefit of U.S. Provisional Application No. 60/556,642 filed 26 Mar. 2004. This application also claims the benefit of U.S. Provisional Application No. 60/622,673 filed 27 Oct. 2004. U.S. Provisional Application No. 60/622,673 and U.S. patent application Ser. No. 10/945,354 are hereby incorporated by reference in their entirety.

### FEDERALLY SPONSORED RESEARCH OR DEVELOPMENT

The subject matter of this application may in part have been funded by the National Institute of Health Grant Nos. 1R21CA102750-01 and 5R21HL071921-02. The government may have certain rights in this invention.

### BACKGROUND

More than 85% of all cancers originate in the epithelia lining the internal surfaces of the human body. The majority of such lesions are readily treatable if diagnosed at an early stage. Recent research on the molecular and cellular alterations in cancerous tissues has provided a better understanding of the mechanisms of the disease. However, these advances have not translated into an improved diagnostic approach for early malignant lesions.

Pathologists qualitatively interpret the histological characteristics such as nuclear atypia (nuclear enlargement, increased variation in nuclear size and shape, increased concentration of chromatin, roughening of the chromatin texture, the margination of nuclear chromatin, etc.) as well as architectural changes throughout the epithelium. Not only do fixation and staining limit the application of histology to the study of the dynamics of disease progression in its natural environment, but also the histological image of a stained tissue sample represents the spatial distribution of the contrast dye, typically hematoxylin and eosin (H&E), which may not be a good representation of the actual cell structure. Therefore, some potentially important diagnostic information may be lost or altered.

Colorectal neoplasms, which originate in the epithelia lining of the colon, are the second-leading cause of cancer deaths in the United States, underscoring the public health imperative for developing novel strategies to combat this malignancy. Screening has been shown to decrease colorectal cancer mortality by both identifying lesions at an early, potentially curable stage and also through prevention of colorectal cancer development by targeting the precursor lesions, the adenomatous polyps. However, there are many barriers to widespread implementation of these strategies, including patient noncompliance, discomfort, economic constraints, resource availability, and risk of complications. Indeed, most eligible subjects do not receive any type of screening for colorectal cancer, which is in marked contrast to screening rates for other common malignancies (e.g., breast, prostate).

Improved screening methodologies are essential to decrease the number of fatalities due to colorectal cancer. Many screening techniques are designed to exploit the “field effect” of colon carcinogenesis, the proposition that the genetic/environmental milieu that results in neoplasia in one region of the colon should be detectable throughout the mucosa. For instance, the detection of distal adenomatous polyps by flexible sigmoidoscopy is commonly used to risk-stratify patients for proximal neoplasia and, hence, the need for colonoscopy. Furthermore, rectal aberrant crypt foci (ACF) have been shown to accurately predict the occurrence of colon adenomas and carcinomas. From a cellular perspective, apoptosis in the uninvolved mucosa (both basal and bile salt induced) has been shown to be a reliable marker for colonic neoplasia. Several biochemical markers have also been evaluated, including colonic protein kinase C activity and mucus disaccharide content.

Although all of these markers have shown a statistically significant correlation between rectal assays and colonic neoplasia (i.e., the field effect), their performance characteristics are suboptimal for clinical practice. For instance, although flexible sigmoidoscopy is a well-established and widely used screening technique, the problems with this test are underscored by the observation that less than one-half of subjects with advanced proximal colon adenomas would also harbor lesions in the sigmoid and rectum. Therefore, flexible sigmoidoscopy would not trigger colonoscopy in these cases and the proximal lesions would have the opportunity to evolve into invasive carcinomas. Thus, the finding of an accurate marker for the field effect would be of major clinical importance.

There are several lines of evidence that subtle perturbations in colonic microarchitecture may be a manifestation of the field effect. For instance, in the “transitional mucosa” (histologically normal epithelium adjacent to colon cancer), a number of abnormalities in the cell nuclei have been noted, including changes in parameters such as total optical density, nuclear area, chromatin texture, and coarseness. Although microarchitectural alterations may serve as an excellent marker of the field effect of colon carcinogenesis, current technology does not allow its practical and accurate detection.

Emerging evidence underscores the critical nature of blood supply augmentation in meeting the metabolic demands of the burgeoning tumor. Indeed, tumor angiogenic markers are important independent prognostic indicator in patients with colorectal cancer (CRC). Their therapeutic implications are highlighted by the demonstration that targeting blood vessel development with the antivascular endothelial growth factor (VEGF) monoclonal antibody bevacizumab resulted in regression in rectal cancer and improved survival in patients with metastatic colorectal malignancies.

While the importance of increased blood supply in CRC development is unequivocal, the stage at which it occurs remains unclear. Angiogenesis has previously been shown as early as small adenomatous polyp or even the ACF stage. Moreover, abnormalities in the microvasculature of the “transitional mucosa” suggest that alterations in blood supply may precede macroscopic neoplastic lesions. These reports are consistent with a variety of malignancies (vulva, cervical, lung, skin, pancreas) that show neoangiogenesis at a predysplastic stage. However, studies in colon carcinogenesis have been suboptimal because of the utilization of semi quantitative determination of microvessel density rather than the technically demanding assessment of mucosal blood content.

Advances in biomedical optics have the potential of enabling real-time in vivo assessment of intracellular structure. Light-scattering spectroscopy (LSS) has been used to identify cellular atypia. The clinical applicability of this technology is indicated by the demonstration that dysplasia in Barrett's esophagus can be accurately identified using an endoscopically compatible LSS probe. LSS was also shown to be able to detect cells undergoing neoplastic transformation in several human organs, including the colon, through evaluation of nuclear size and chromatin density, as well as early stages of colorectal carcinogenesis. However, this relatively basic technology relies on detection of altered nuclear size and chromatin content, and therefore it may be less adept at detecting the more subtle microarchitectural changes of the field effect and thus less useful in screening for colorectal cancer.

There are two principal methods to study elastic light scattering: measuring the (1) angular and (2) spectral distributions of the scattered light. In the first approach, the illumination wavelength is fixed and the angular distribution of the scattering light l(λ) is recorded with a goniometer. In the second approach, the object is illuminated by a broadband light source and the spectrum of the scattered light l(θ) for either a specific scattering angle or integrated over a certain angular range is measured. In addition, by measuring light-scattering spectra at different scattering angles, the size distribution of particles smaller or larger than the wavelength can be obtained.

Several other optical techniques have been used to detect cells. Bio-optics techniques (optical coherence tomography, Raman spectroscopy, angle resolved low-coherence interferometry, and so on) have been shown to be useful in detecting pathologically apparent dysplasia. However, previous investigations using these techniques have focused on the diagnosis of more advanced, histologically apparent stages of neoplastic transformation and none of these techniques have been shown to allow identification of predysplastic epithelium.

The proliferation of smooth muscle cells (SMCs), central to the cardiovascular disease, is a characteristic feature in arteries of hypertensive patients and animals. Therefore, there has been significant interest in defining both positive and negative regulators of SMC growth: laminin and fibronectin are the extracellular matrix substrates and have been well identified and characterized as the normal regulators of SMC differentiation. It has been shown that fibronectin promotes the transition of arterial SMCs from a contractile to a synthetic phenotype, accompanying the loss of myofilaments and outgrowth of an extensive endoplasmic reticulum and a large Golgi complex. Moreover, the characterization of cellular interactions with a biomaterial surface is important to the development of novel biomaterials and bioengineered tissues. Current techniques to characterize the cell adhesion and phenotypic differentiation are destructive, complicated, expensive and time-consuming and do not allow in situ quantitative assessment.

Light scattering has been used as a tool for polymer characterization for many years. For example, laser light scattering was used as a non invasive, sensitive analytical method in the characterization of polymers and colloids in solution. Small-angle X-ray scattering (SAXS) and wide-angle X-ray scattering (WAXS) measurements are used for morphological investigations of crystalline polymers. Light scattering is also a routine method used for molecular weight and size distribution measurements. Current state-of-the-art light-scattering techniques for polymer characterization are limited to polymers that can be dissolved in solution eliminating their use for crosslinked polymer systems. To date, there are no reports regarding the use of light scattering to characterize the molecular weight or mechanical properties of polymeric materials in solid state.

### BRIEF SUMMARY

In a first aspect, the present invention is a method of examining a sample, comprising measuring, as function of wavelength of light elastically scattered from the sample, at least 2 properties, selected from the group consisting of scattering angle theta of the light, scattering angle phi of the light, and polarization of the light. The scattering angle theta is an angle between the backward direction and the direction of propagation of the light, and scattering angle phi is an angle between the incident light polarization and the projection of the direction of the light propagation onto a plane in which the incident electric field oscillates.

In a second aspect, the present invention is a multi-dimensional elastic light scattering instrument, comprising (i) a light delivery system, for delivering a collimated linearly polarized beam of light to a sample, (ii) a light collection system, for collecting light from the light delivery system scattered from the sample, and (iii) optionally, a calibration system. The instrument measures, as function of wavelength of light elastically scattered from the sample, the scattering angle theta of the light, the scattering angle phi of the light, and the polarization of the light. The scattering angle theta is an angle between the backward direction and the direction of propagation of the light, and the scattering angle phi is an angle between the incident light polarization and the projection of the direction of the light propagation onto a plane in which the incident electric field oscillates.

In a third aspect, the present invention is a multi-dimensional elastic light scattering probe, comprising (a) a first optical fiber, (b) a first set of at least one optical fiber, and (c) a second set of at least one optical fiber. The first optical fiber, the first set, and the second set, all have an end optically coupled to an end of the probe, and the probe has an outer diameter of at most 1.5 mm.

### BRIEF DESCRIPTION OF THE DRAWINGS

FIG. 1 illustrates a multi-dimensional elastic light scattering (MD-ELF) instrument.

FIGS. 2A and B illustrate a probe.

FIGS. 3 A, B, C and D are light scattering fingerprints: (A) measured fingerprint for 5.8 μm polystyrene microspheres; (B) calculated fingerprint for 5.8 μm polystyrene microspheres; (C) measured fingerprint for 9.2 μm polystyrene microspheres; and (D) calculated measured fingerprint for 9.2 μm polystyrene microspheres.

FIGS. 4A, B, C and D are light scattering fingerprints of precancerous rat colon tissues early in carcinogenesis: (A) saline-treated rat, proximal colon; (B) AOM-treated rat, proximal colon; (C) saline-treated rat, distal colon; and (D) AOM-treated rat, distal colon.

FIGS. 5(a), (b), (c) and (d) are graphs of the temporal progression of light scattering markers of early carcinogenesis in the colon: (a) spectral slope of the distal colon; (b) fractal dimension of the distal colon; (c) spectral slope of the proximal colon; and (d) fractal dimension of the proximal.

FIG. 6 is a graph of PC1 obtained from control and AOM-treated rat distal colon tissues at 2, 5, 6, 12, and 20 weeks after injection of carcinogen.

FIG. 7 is a light scattering fingerprints from crosslinked POC films post-polymerized under different conditions (Left: 80° C., no vacuum, 2 days; Right: 80° C., no vacuum, 14 days; the scale represents the intensity of backscattering light.

FIG. 8A is a graph of the spectra of the average intensity vs. wavelength (LS1: 80° C., no vacuum, 2 days; LS10: 80° C., no vacuum, 14 days).

FIG. 8B is a graph of the normal equivalent size distribution of POC (LS1: 80° C., no vacuum, 2 days; LS4: 120° C., vac, 2 days; LS5: 120° C., vac, 3 days; LS6: 140° C., vac, 2 days).

FIGS. 9A-F are graphs of the linear fit of spectral slope and equivalent size to logarithm of molecular weight between crosslinks (A and B), Young's modulus (C and D), and tensile stress (E and F) of POC respectively (data is expressed as mean value±standard error of mean).

FIGS. 10A and B are graphs of the linear fit of spectra slope (A) and equivalent size (B) to volume change of POC in DMSO (data is expressed as mean value±standard error of mean).

FIGS. 11A-F are graphs of the linear fit of spectra slope and equivalent size to logarithm of molecular weight between crosslinks (A and B), Young's modulus (C and D), and tensile stress (E and F) of PGS respectively (data is expressed as mean value±standard error of mean).

FIGS. 12A and B are graphs of the linear fit of spectra slope (A) and equivalent size (B) to volume change of PGS in DMSO (data is expressed as mean value±standard error of mean).

FIGS. 13A-C are graphs of the linear fit of spectral slope to logarithm of molecular weight between crosslinks (A), tensile stress (B) and Young's modulus (C) of polystyrene respectively (data is expressed as mean value±standard error of mean).

FIG. 14 is a graph of size distributions of SMCs' cellular and subcellular structures grown on two different substrates: laminin and fibronectin.

FIG. 15 is a graph of changes of spectral slope for SMCs grown on two different substrates: laminin and fibronectin.

FIG. 16 is a graph of principal Component Analysis (PCA) Principal Component 2 (PC2) of light scattering fingerprints obtained from 4D-ELF's collected for SMCs grown on fibronectin and laminin.

FIGS. 17A and B are graphs demonstrating that four dimensional elastic light scattering fingerprinting accurately measures mucosal and mucosal/submucosal blood content: (A) mucosal and (B) mucosali/submucosal models.

FIGS. 18A-C are graphs demonstrating that an increase in blood content is one of the earliest events in neoplastic transformation in the azoxymethane (AOM) treated rat model: (A) representative light scattering spectra recorded from colonic superficial mucosa and mucosa/submucosa of rats treated with AOM (two weeks post-AOM treatment); (B) mucosal/submucosal blood content was increased in the distal colon at two weeks post-AOM injection, a time point that precedes aberrant crypt foci or other conventional markers of neoplasia; and (C) superficial blood content.

FIGS. 19A-C are graphs demonstrating that the temporal and spatial nature of augmentation of colonic mucosal/submucosal blood content is consonant with progression of carcinogenesis in the azoxymethane (AOM) treated rat model: (A) aberrant crypt foci (ACF) analysis was performed using the technique described in the methods section; (B) in the distal colon, there was a progressive and statistically highly significant increase in blood content over time (ANOVA, p value ,0.0001); and (C) in the proximal colon, there was a marginal increase in blood content (p=0.12), paralleling the minimal carcinogenic effect of AOM in this region of the colon, as noted in (A).

FIGS. 20A and B are graphs demonstrating that the blood content increase in the MIN mouse model: (A) mucosal/submucosal blood content was significantly increased in the small bowel but not in the colon; and (B) the number density of superficial red blood cells (RBCs) (1/mm2) paralleled findings in the mucosa/submucosa in that there was a significant increase in the small bowel but not in the colon.

FIG. 21 is a graph which provides evidence of early increase in blood supply in human colon carcinogenesis

### DETAILED DESCRIPTION

Multi-dimensional elastic light scattering (MD-ELF) allows acquisition of light-scattering data in several dimensions. The dimensions of MD-ELF include (1) wavelength of light λ, (2) the scattering angle θ (i.e., the angle between the backward direction and the direction of the propagation of scattered light), (3) azimuthal angle of scattering Φ (i.e., the angle between the incident light polarization and the projection of the direction of the scattered light propagation onto the plane in which the incident electric field oscillates), and (4) polarization of scattered light. When all four dimensions are used the MD-ELF may be referred to as 4D-ELF, in which scattered light is analyzed as a function of its wavelength in dimension 1, direction of propagation in dimensions 2 and 3, and polarization in dimension 4.

The present invention makes use of the discovery that MD-ELF is able to accurately detect changes in the colon, which correlate well with carcinogenic progression, and therefore may be used for colon cancer screening. MD-ELF is able to detect these changes far earlier than previously described markers. The data collected using MD-ELF may be analyzed by a variety of techniques: fingerprint analysis, spectral analysis, spectral slope, fractal dimension, and principal component analysis (PCA).

Four D-ELF is also able to provide quantitative information about biological structures without the need for cell fixation, staining, or other processing, and enables probing of cellular and subcellular organization at scales from tens of nanometers to microns, thus encompassing a spectrum of structures ranging from macromolecular complexes to whole cells. Light reflected from a tissue after only few scattering events (i.e. “single scattering component”) is extremely sensitive to tissue microarchitecture and, typically, probes only the superficial tissue. In 4D-ELF this is accomplished via polarization gating. The differential polarization signal (Δ|=|_{∥}−|⊥), is primarily contributed by the most superficial tissue structures. The copolarized signal |_{∥}, diffuse reflectance signal |_{∥}+|⊥, and the cross-polarized signal Ii provide information about progressively deeper tissues (up to several millimeters below the surface). Four D-ELF is able to detect the structural difference of SMCs grown on different substrates, and potentially characterize the cell/biomaterial interactions. Additional details of this study may be found in Liu, Y, et al. “Light scattering ‘fingerprinting’ for characterization of smooth muscle cell proliferation” Advanced Biomedical and Clinical Diagnostic Systems II. Edited by Cohn, Gerald E., et al. Proceedings of the SPIE, Volume 5319, pp. 32-40 (2004), the entire contents of which are hereby incorporated by reference.

Four D-ELF may also be used to detect morphological changes within a polymer network at the nano- to micro-scale, enabling non-invasive and quantitative characterization. It is advantageous because it is non-destructive to the polymer, it provides a real-time analysis that is quantitative, and this information is obtained from the solid state polymer.

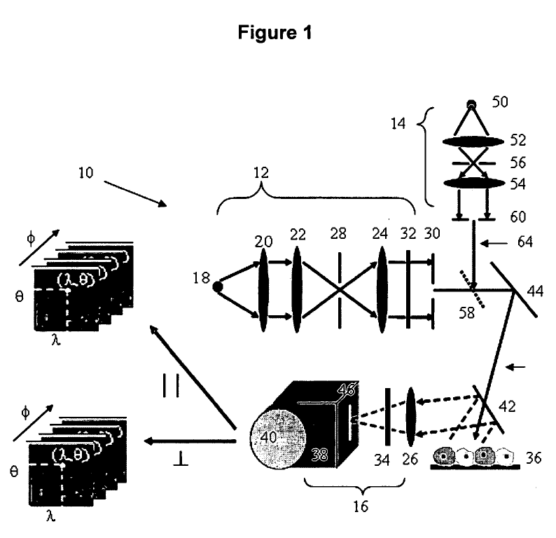

FIG. 1 illustrates an MD-ELF instrument 10, which includes a light delivery system 12, a light collection system 16, and an optional calibration system 14. The light delivery system delivers a collimated linearly polarized beam of light 62 to a sample on a sample stage 36, and includes, for example, a light source 18, optically coupled to a condenser 20, a first lens 22, a first aperture 28, a second lens 24, a first polarizer 32, and a second aperture 30. To assist in directing the light to the sample and sample stage a mirror 44 may be used. The sample scatters the light, which is then collected and recorded by the light collection system, which includes, for example, a third lens 26, optically coupled to a second polarizer 34, a spectrograph 38 (which includes a slit 46), and a light recorder 40. To assist in collecting the scattered light, a beam splitter 42 may be used. The calibration system includes, for example, a calibration light source 50, optically coupled to a first calibration lens 52, a first calibration aperture 56, a second calibration lens 54, and a second calibration aperture 60 (which may each be the same as the first lens 22, the first aperture 28, the second lens 24, and the second aperture 30, respectively) to produce a collimated calibration beam of light 64. The instrument may also include a movable mirror 58 for directing the collimated calibration beam of light to the sample.

Preferably, the lens 26 is positioned one focal distance from the slit of the spectrograph, so that an angular distribution of the scattered light is projected onto the slit. Preferably, the spectrograph diverts the light according to wavelength, in a direction orthogonal to the slit, projecting it onto the light recorder. This allows the light recorder to record the intensity of the light for various wavelengths and angles of scattering. The azimuth of scattering may be selected by rotating the first polarizer 32. Since the first and second polarizers may be moved independently, measurement of the intensity of 2 independent components of the light scattered from the sample may be measured: scattered light polarized along the direction of polarization of the incident light (the co-polarized component l_{∥}) and scattered light polarized orthogonally to the polarization of the incident light (the cross-polarized component l_{⊥}).

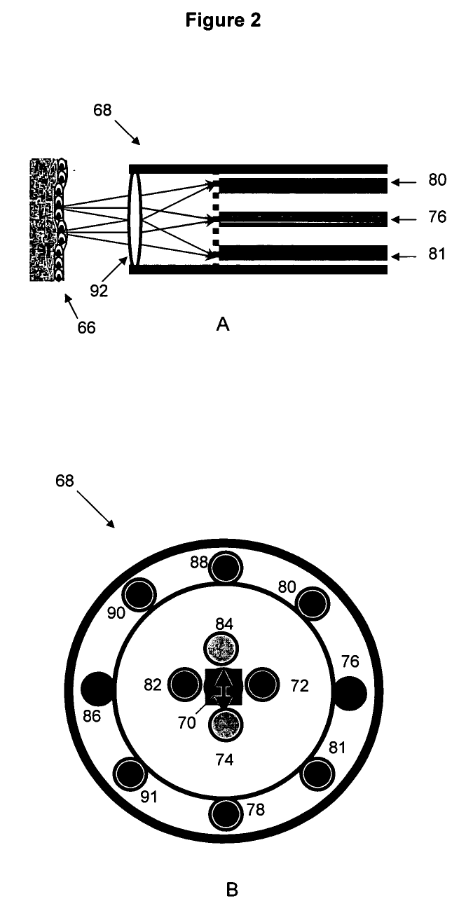

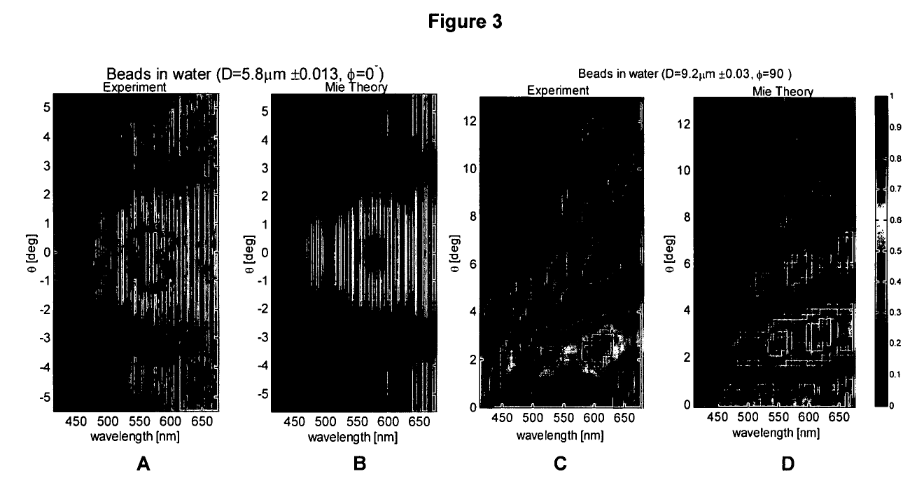

FIGS. 2A and 2B illustrate an example of a probe 68, which may be included as part of the light delivery and the light collection systems (and optionally, the calibration system), allowing for examination of tissue 66 in vivo. The probe contains multi-mode fibers (72-91) for bringing light elastically scattered by the tissue to the spectrometer, positioned in concentric rings around central fiber 70, which delivers light from the light source. The tip of the delivery fiber and half of the collection fibers (72-81) will be coated with a polarizing thin film to linearly polarize the emerging light and collect co-polarized component of the scattered light l_{∥}. The other half of the collection fibers (82-91) will be coated to collect cross-polarized light l_{⊥}. On the collection end of the probe, the fibers form a line and are optically coupled to the spectrometer. The spectra collected by each channel will be recorded independently and simultaneously. Positive antireflection-coated aberration-corrected GRIN lens 92 is preferably positioned one focal distance from the fiber tips and will collimate light emerging from fiber 70. Moreover, analogous to the third lens in the MD-ELF instrument, lens 92 focuses light backscattered by the tissue 66 onto different fibers of the probe depending on the angle of scattering (for example, ˜1° for the first ring and ˜5° for the second ring). Furthermore, both l_{∥} and l_{⊥} are collected, allowing polarization gating, analogous to the polarization gating in the system illustrated in FIG. 1. In the probe illustrated in FIGS. 2A and 2B, possible characteristics are: fiber NA—0.11; fiber diameter—50 μm; focal length of lens 92—3 mm; distances between fiber 70; the first fiber ring (including fibers 72, 74, 82 and 84), and the second fiber ring (including fibers 76-81 and 86-91)—0.075 and 0.25 mm, respectively; outer diameter of the probe 68<1.5 mm. This scheme insures that the probe can fit into the accessory channel of a colonoscope. Other configurations are possible, such as 3, 4 or more rings of fibers; and 4, 8, 12, 16, 20, 32, or more fibers in each ring.

To show the feasibility of using the information provided by the spectral-angular maps to study the initial stages of carcinogenesis, studies were conducted involving an animal model of colon cancer. The azoxymethan (AOM)-treated rat is an established, robust, and well-validated model of human colon carcinogenesis and replicates the progression of the genetic, cellular, and morphologica events of human sporadic colon cancer. When Fisher rats are treated with AOM, a colon-specific carcinogen, aberrant crypt foci (ACF) develop within 5-10 weeks after AOM injection. The appearance of ACF is the earliest detectable biomarker of colon carcinogenesis; however, recent reports have suggested that some genetic events may precede the development of ACF. The cellular correlates of genetic and epigenetic changes include inhibition of apoptosis, allowing the otherwise short-lived colonocytes to accumulate requisite mutations for neoplastic transformation and increased proliferation, allowing clonal expansion of initiated cells. It must be emphasized that these critical initial cellular and genetic events have no currently identifiable morphological correlates; thus, with the current armamentarium, these lesions are impossible to diagnose. The development of technologies to detect these lesions would be of considerable clinical importance given the field effect of colon carcinogenesis. Assessment of early lesions in the distal, more accessible colon may provide accurate risk-stratification for more invasive procedures.

In order to assess the sensitivity and utility of 4D-ELF for the detection of cancer, we therefore used the AOM colon cancer model and focused on time-points during carcinogenesis where no current biomarkers are available. Specifically, Fisher rats received either 2 weekly injections of AOM or saline. The rats were killed at various times after the second injection, their colons divided into proximal and distal segments, and the segments were examined by MD-ELF (4D-ELF). The number of ACF on a subset of animals was analyzed in this study to correlate this well-validated biomarker of colon carcinogenesis to the 4D-ELF readings. ACF were detectable at week 4 and progressively increased in both number and complexity over the course of the experiment. There was a marked distal predominance in ACF. Although proximal ACF occurred, these required longer to develop and were less numerous than distal ACF. No ACF were detected in the saline-treated animals.

To analyze the 4D-ELF data, a variety of parameters that span the spectrum of microarchitectural abnormalities were assayed. Fingerprint analysis gives a dramatic, albeit qualitative, appreciation of AOM-induced alterations. The spectral slope analysis evaluates size distribution of particles ranging from macromolecules to organelles. Fractal dimension, on the other hand, reflects alterations of the tissue organization at much larger scales, ranging from large organelles to groups of cells. PCA is a standard data procedure for assessing underlying structure in a data set. To infer a relationship to colon carcinogenesis, we correlated the 4D-ELF signatures with the subsequent occurrence of ACF. Specifically, neoplastic signatures should progress over time and be predominantly in the distal colon, especially early during carcinogenesis (mirroring the ACF data). All data from AOM-related signatures were compared with an age-matched saline-treated rat.

Whether 4D-ELF would be able to detect the field effect of colon carcinogenesis was assessed. 4D-ELF is able to accurately identify alterations in the colonic mucosa at a far earlier stage than any previously described markers. Furthermore, these changes correlated well with the carcinogenic progression in this model. Four D-ELF may be used for colon cancer screening because of its remarkable sensitivity to the earliest changes in carcinogenesis. Using quantitative analysis of tissue microarchitecture, MD-ELF can detect the earliest alterations in neoplastic transformation (2 weeks after carcinogen treatment in the animal model studied).

The relevance of these 4D-ELF changes to carcinogenesis is supported by both the temporal and spatial correlation. Temporally, the marked alterations detected at week 2 progressively increased in magnitude over time consonant with the neoplastic effects of azoxymethane (AOM) in this model. Spatially, the early signature alterations were predominantly in the distal colon, the region of the colon most susceptible to ACF and tumor development. Moreover, the changes noted with 4D-ELF occurred at 2 weeks after treatment with AOM, a time point far earlier than seen with other conventional biomarkers. This time point was of particular importance in that the nonspecific genetic and cellular changes associated with acute carcinogen administration have dissipated. Therefore, alterations at this time reflect the earliest changes related to the field effect of carcinogenesis. The biological plausibility of this previously undescribed microarchitectural change is supported by several recent reports cataloging genetic changes in colon carcinogenesis. Indeed, one study reported that 4 weeks after treatment with AOM, a decrease in APC message was detectable with a concomitant increase in cyclooxygenase 2 and c-myc expression. Although the architectural consequences of these genetic alterations were not explored, APC, c-myc, and cyclooxygenase 2 have been reported to alter cellular structure and function.

The data indicate that the microarchitectural perturbations in the histologically normal mucosa identified by 4D-ELF represent a reliable marker of the field effect of colon carcinogenesis. However, as opposed to classic definitions of the field effect, the alterations noted occurred before onset of neoplasia. This has great clinical utility to accurately identifying individuals at future risk of developing colorectal cancer, and quantifying each individual's risk for developing neoplasms. A possible explanation is that 4D-ELF may be detecting previously undescribed preneoplastic lesions, although such putative lesions would have to be remarkably abundant.

The microarchitectural changes that we noted early in colon carcinogenesis encompassed a large spectrum of parameters. The results indicate that the size distribution of submicron intraepithelial structures shifts toward larger sizes very early in carcinogenesis. Although the biological determinants of this phenomenon are unclear, it may reflect an increase in the sizes of macromolecular complexes (for example, more protein-protein interactions). Fractal dimension, on the other hand, reflects changes in cell organization at much larger scales, ranging from large organelles to cells. Alterations in fractal dimension have been postulated to be one of the earliest changes in colon cancer. The most common way of measuring fractal dimension is through box-counting approximations, which would not be practical for colon cancer screening.

The data generated by 4D-ELF were also analyzed through principal component analysis (PCA). PCA has been used for many biological and clinical purposes, including both assessment of karyotypic alterations and distinct biological features (e.g., global molecular phenotype) in human colon cancer. This variable reduction procedure is useful in assessing underlying structure in a complex data set. Because principal components are extracted in a stepwise fashion, the first principal component is responsible for the largest amount of the variance. It has now been discovered that principal component 1 (PC1) is a marker of the field effect that may be exploited for colorectal cancer screening.

The data obtained from light-scattering fingerprinting should not be considered a mere substitution for the morphologic tissue analysis using light microscopy. The 4-dimensional information extracted from ELF provides much greater biological insights than the previously used technologies. The critical advantages are related to the quantitative information regarding nanoscale architecture on living tissues. Four D-ELF gives information at the level of electron microscopy and yet keeps the levels of cellular organization that may be lost with staining and fixation, allowing heretofore-undiscovered insights regarding microarchitectural changes that occur early in neoplastic transformation. Given the complexity of the signatures, some signals may not allow direct correlation to a specific feature of the cellular architecture but still may serve as valuable intermediate biomarkers for carcinogenesis.

Studies using the other major experimental model, the multiple intestinal neoplasia (MIN) mouse, also noted marked 4D-ELF alterations occurring at the pretumorigenic stage, dispelling the possibility that these findings are model specific (Roy, H., et al. Cancer Epidemiol. Biomarkers Prev. 2005;14(7) (July 2005); and Roy, H., et al. Mol. Cancer Ther. 2004; 3(9) (September 2004); the entire contents of both of these references are hereby incorporated by reference).

Four D-ELF allows us to obtain quantitative information about the microvasculature in tissue samples by analyzing the characteristic absorption/reflection spectra of red blood cells (RBC). The accuracy and sensitivity of this technique in determining the blood content far exceeds other non-optic techniques previously utilized. Thus 4D-ELF is perfectly suited to investigate changes in blood content in early carcinogenesis.

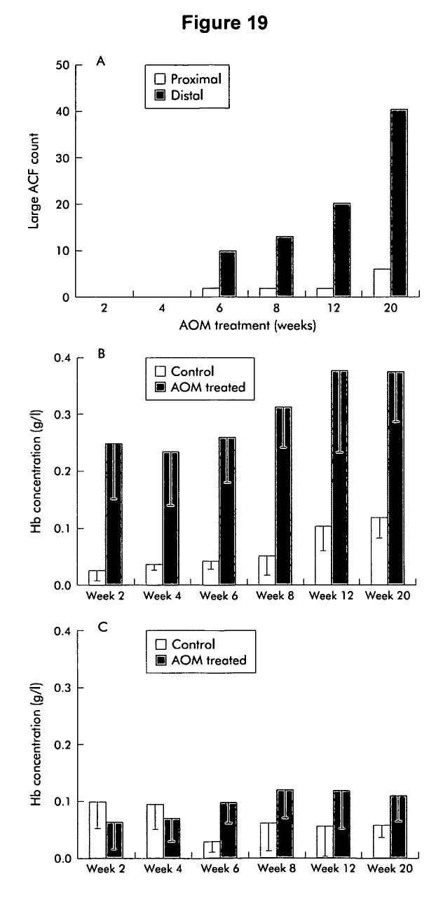

We used 4D-ELF to probe the microvasculature in the uninvolved colonic mucosa of AOM treated rats. We were particularly interested in evaluating blood supply changes at two weeks post AOM when ACF are undetected whereas the non-specific carcinogen effects have dissipated (from a temporal perspective, large ACF were detectable at six weeks and increased in number over time (FIG. 19A)). We detected an early increase in blood supply (EIBS) at the premalignant stage of colon carcinogenesis. These changes increased in magnitude over time, in a manner consonant with neoplastic transformation. We also utilized immunoblot analysis of mucosal scrapings for hemoglobin to confirm our 4D-ELF findings (albeit with considerably less sensitivity). Furthermore, we replicated these results in another animal model of colon carcinogenesis, the MIN mouse. Finally, in order to show relevance of EIBS to human colon carcinogenesis, we performed a pilot colonoscopic biopsy study.

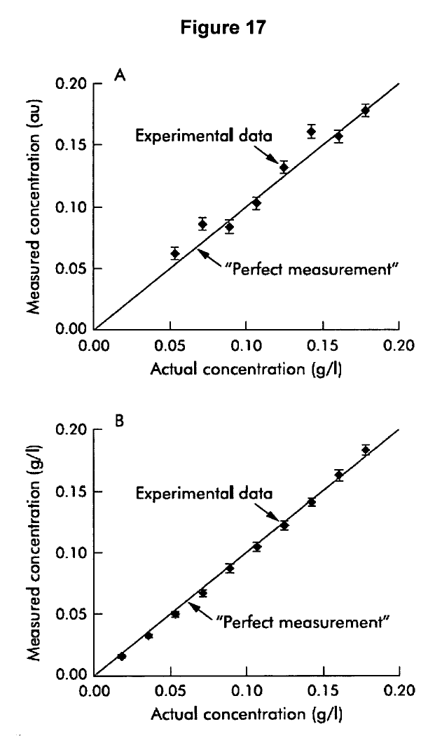

FIG. 17 compares hemoglobin concentrations obtained using the optical measurements with the actual values within the physiological range. As evident from FIG. 17A, our technique enabled measurement of blood content with excellent accuracy (error <3.6%). As demonstrated in FIG. 17B, our technique provided outstanding accuracy, with error <1.8%. These performance characteristics are superior to all other conventional techniques in measuring blood content in tissue.

The phenomenon of EIBS lends itself to potential applications in CRC screening and prevention. From a screening perspective, our data show that even at the earliest time point (two weeks post AOM injection) increased blood supply was able to detect carcinogen exposure with a sensitivity of 93.8%, specificity of 95.8%, and a positive predictive value of 96.8%. Our human data support the clinical relevance of the early increase in blood supply.

Additional details of this study may be found in Wali, R K, et al. “Increased microvascular blood content is an early event in colon carcinogenesis” Gut 2005; 54:645-660, the entire contents of which are hereby incorporated by reference.

The success of MD-ELF in the detection of colon cancer indicates that it may also be useful for the detection of early, previously undetectable stages of precancerous lesions in other endoscopically or laparoscopically accessible organs, such as the esophagus, stomach, bladder, oral cavity, cervix, ovary, pancreas, etc.

For the 4D-ELF measurements and analysis of a polymer, 10 to 15 random measurements were taken from each sample. The slope of the intensity versus wavelength spectra was obtained for correlation to mechanical and molecular weight data. Computational spectra derived from Mie Theory were fitted to the differential polarization polymer spectra to obtain size distribution of scattering structures. Sizes were correlated to mechanical and molecular weight data. The data obtained from these studies and the conclusions that may be reached are discussed further in Example D.

From these analyses it has been determined that there are intrinsic structural characteristics of polymers that can be correlated to extent of reaction and mechanical properties. Further, these characteristics may be assessed in a non-perturbing, real time and quantitative manner using the 4D-ELF technique. The 4D-ELF can detect morphological structures within solid polymeric materials, which can be used to assess the extent of reaction and mechanical characteristics. There was a linear correlation between spectral slope (and equivalent size of scattering structure) and: (1) log of molecular weight between cross links (2) Young's modulus and tensile strength, and (3) log of molecular weight.

### EXAMPLES

A. MD-ELF

The MD-ELF instrument used included the following (with reference to corresponding parts shown in FIG. 1): A broadband light from a 75 W Xenon arc lamp 18 (Oriel, Inc., Stratford, Conn.) was collimated by a condenser 20 (f/1, two element fused silica, Oriel, Inc., CT) and a 4-f relay system including lenses 22 (achromat, f=160 mm, D=mm, Melles Griot, Irvine, Calif.), 24 (achromat, f=300 mm, D=50 mm, Melles Griot, CA), and an aperture 28. The resulting beam had a divergence of 0.2°. This beam was polarized by a polarizer 32 (Dichroic sheet polarizer, Melles Griot, CA) and its diameter was reduced to 1 mm by a field diaphragm 30. A mirror 44 deflected the beam through the beamsplitter 42 (broadband nonpolarizing, Newport, Calif.) onto the sample, which was mounted on a sample stage 36. To avoid the specular reflection from tissue surface, the incident beam was orientated at an angle of 15° to the normal to the sample surface. The light scattered by the sample was collected by a lens 26 (f=31 mm, D=17.5 mm, Melles Griot, CA). A polarizer 34 selected the polarization state of the scattered light so that the co-polarized component (∥) and the cross-polarized component (⊥) of the scattered light could be recorded independently. An entrance slit of a spectrograph 38 (SpectraPro-150, Acton Research Corp., Acton, Mass.) was placed in the focal plane of the lens 26. This spectrograph was coupled with a charge-coupled device (CCD) camera 40 (CoolSnapHQ, Roper Scientific Inc., Trenton, N.J.). The spectrograph was positioned such that the slit was at a focal distance from the lens 26. Therefore, all scattered rays with an identical scattering angle θ and an azimuthal angle Φ were focused into a point on the entrance slit. An angular distribution of the scattered light was projected onto the slit of the spectrograph. For example, the scattering in the backward direction was mapped at the center of the slit. The azimuthal angle Φ was defined by the angle between the direction of the spectrograph slit and the polarization direction of the incident beam, which was selected by rotating the polarizer 32. The co-polarized intensity (l_{∥}) and the cross-polarized intensity (l_{⊥}) were measured by rotating the polarizer 34 parallel and perpendicular to the polarizer 32, respectively. The spectrograph spread the light in the direction perpendicular to the slit according to its wavelengths. Thus, the CCD recorded a matrix of the scattered intensities, where one axis corresponded to the wavelength of light λ and the other to the angle of scattering θ for a fixed azimuthal angle Φ and a polarization state (∥ or ⊥). These maps were collected for three azimuthal angles Φ=0°, 45°, and 90°, in the spectral range from 400 to 700 nm, and for the scattering angles θ ranging from 0° to 12°. After the co-polarized intensity maps l₈₁(λ,θ) and the cross-polarized intensity maps l_{⊥}(λ,θ) were collected for θ=0, 45°, and 90°, the sample was removed and the background intensities were measured for each Φ and subtracted from the measured intensities to remove stray illumination components and background noise to obtain l_{∥} and l_{⊥}. These maps were normalized by the respective intensity maps l_{∥}^{Xe} and l_{⊥}^{Xe} collected from a reflectance standard (Ocean Optics, Inc., Dunedin, Fla.) to account for the nonuniform spectrum of the xenon lamp illumination and other artifacts. Then, the differential polarization intensity maps were calculated as Δl=l_{∥}/l_{∥}^{Xe}−l_{⊥/l}_{⊥}^{Xe}. For spectrograph calibration, a mercury lamp 50 (Ocean Optics, Inc., FL) was used. The calibration beam was collimated and impinged upon a mirror 58, which was mounted on a flipper (New Focus, San Jose, Calif.). Depending on the orientation of the flipper, either the xenon or the mercury light beams reached the sample stage. The calibration beam was reflected by the reflectance standard and collected by the spectrograph. Thus, the position of the spectrograph grating was calibrated with the emission lines of the mercury lamp.

B. Tissue Phantoms

The instrument was tested and calibrated with tissue phantom consisting of the aqueous suspensions of polystyrene microspheres (refractive index n=1.59) (Polyscience, Inc., Warrington, Pa.) of various diameters ranging from 1 μm to 10 μm. The first purpose of these experiments was to study the efficacy of the polarization gating for the decoupling of the single and multiple scattering components of the returned signal. The number density of the microspheres was increased and the scattering coefficient μₛ was calculated using Mie theory. The optical thickness τ of the tissue phantom was varied from 0 to 5.5 (τ=μₛz, where z is the physical depth of the medium; light traversing a medium with τ=1 undergoes, on average, one scattering). The co-polarized signal (l_{∥}) and the cross-polarized signal (l_{⊥}) were recorded at the three azimuthal angles and the differential polarization intensity (Δl) was calculated by subtracting l_{⊥} from l_{∥}. Also, the DOP was calculated from the same data. The second purpose of these experiments was to ensure the proper calibration of the instrument. To achieve this, we compared the angular, azimuthal, and spectral distributions of the scattered signals with those simulated using Mie theory. The spectral distributions at several fixed scattering angles and the angular distributions at several fixed wavelengths were compared with Mie theory for all azimuthal angles.

C. Colon Carcinogenesis

The AOM animal model has been the most widely used animal model over the last decade for studying colon carcinogenesis and chemopreventive agents. Several ongoing nutritional and chemopreventive trials in human colon cancer are, in part, based on the results generated using the AOM model. To date, no side effects of AOM that are not directly related to carcinogenesis have been established. The AOM model is the most robust animal model because of the strong similarities in the morphological, genetic, and epigenetic alterations with human colon carcinogenesis. The same molecular and biochemical markers, such as K-ras, AKT, β-catenin, PKC, MAP kinse, and aberrant crypt foci (ACF) in human cancer are identically activated in the AOM model. For example, ACF are precursor lesions, which are observed on the colonic mucosal surface of the AOM model and human cancer. A small proportion of ACF develop dysplasia, evolve into adenomas, and some adenomas eventually degenerate into carcinomas. Adenomas and adenocarcinomas typically are detectable 20-30 weeks after the AOM injection. Both the ACF and tumors show distal colon predominance, further mirroring human sporadic colon cancers. An increased blood supply due to neovascularization (i.e., angiogenesis) of mucosal and submucosal tissues is observed approximately 40 weeks after AOM administration. At a genetic level, AOM leads to the production of O⁶-methylguanine residues in the DNA resulting in mutations of a variety of genes, including β-catenin and K-ras, and overexpression of AKT and epidermal growth factor receptor activation.

All animal studies were performed in accordance with the institutional Animal Care and Use Committee of Evanston-Northwestern Healthcare. Forty-eight male Fisher 344 rats (150-200 g) were randomized equally to groups that received either 2 weekly intraperitoneal injections of AOM (15 mg/kg) (Sigma Chemical Co., St. Louis, Mo.) or saline. Rats were fed standard chow and were killed at various times after the second injection (2, 4, 5, 6, 8, 12, and 20 weeks). Colons were removed, flushed with phosphate-buffered saline, and divided into equal proximal and distal segments. Four D-ELF analysis was performed on fresh tissue. Quantitation of ACF was performed on a subset of animals using methods previously described: after fixation overnight in 10% buffered formalin, colon segments were stained for 2 minutes in 0.2% methylene blue (Sigma Chemical Co.), rinsed in phosphate-buffered saline, and examined with a dissecting microscope. ACF (defined as a foci containing ≧2 crypts) were scored by an observer blinded to treatment.

I. Analysis of Light-Scattering Fingerprints

Four-dimensional light-scattering fingerprints contain a wealth of information about tissue microarchitecture and nanoarchitecture. A number of light-scattering signatures can be linked to specific properties of cell architecture, including the size distribution of intraepithelial nanoscale and microscale structures (from ˜30-40 to 800 nm) and the fractal dimension of the cell structure at supramicro scales (greater than ˜1 μm). The combination of these measures enables quantitative characterization of epithelial architecture in a wide range of scales, from tens of nanometers to microns.

To obtain the complete size distribution of subcellular structures at each tissue site, the spectra computationally simulated using Mie theory were fit to the differential polarization tissue spectra for a given scattering angle and azimuth of scattering using the conventional least-squares minimization algorithm. In each fitting, several types of size distributions (normal, log-normal, or uniform) were assumed. It was found that the spectra recorded by the instrument for scattering angles within ±5° from the backward direction had spectral behavior similar to an inverse power-law, which is consistent with previous results. These studies confirmed that if the sizes of scatterers are widely distributed, as is characteristic of biological tissues, the log-normal or power-law size distributions provide fits superior to those obtained using a normal or uniform size distribution. This agrees well with observation. The log-normal probability distribution depends on 2 parameters: its mean (i.e., the mean size of tissue structures giving rise to the scattering signal) and the standard deviation of particle sizes, which characterizes particle size variability. Therefore, these parameters were varied to minimize the x². The size-sensitivity studies showed that the differential polarization spectra are primarily sensitive only to scatterers with sizes ranging from 40 to 800 nm. Therefore, these limits provide the range of validity of the size distributions obtained using the fitting algorithm.

Principal component analysis (PCA) was also used as one of the tools for data analysis. For PCA, the light-scattering spectra were averaged over scattering angles from −5° to 5°. Each spectrum was preprocessed by mean scaling. A data matrix was created in which each row of the matrix contained the preprocessed spectrum measurement and each column contained the preprocessed scattering intensity at each wavelength. The scores of all principal components were calculated using Matlab statistics toolbox software version 6.5 (The Mathworks, Inc., Natick, Mass.).

II. Elastic Light-Scattering Fingerprints

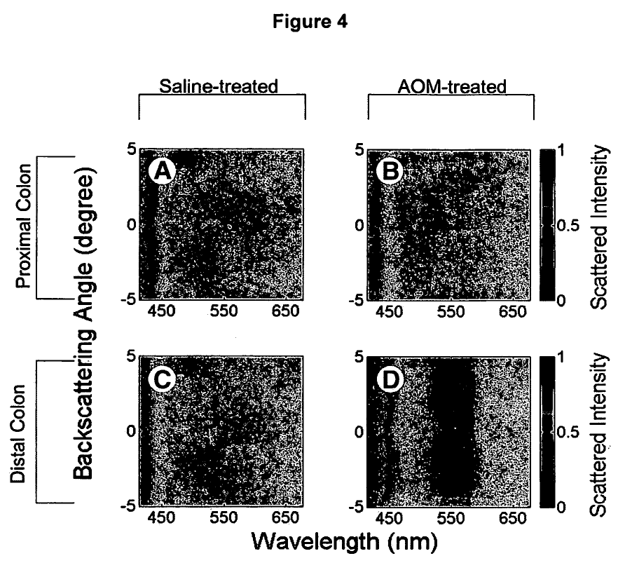

FIG. 4 shows representative light-scattering fingerprints recorded from a rat at an early stage of carcinogenesis (2 weeks after carcinogen treatment) and a control animal (2 weeks after saline treatment). For the control (saline-treated) animal, there are slight differences between the back-scattering intensity (especially at larger back-scattering angles) as indicated by subtle changes in color intensity recorded from proximal (FIG. 4A) and distal (FIG. 4C) colons, respectively. This finding is consistent with the biological differences in the regions of the colon. Moreover, in the proximal colon (FIG. 4B), treatment with AOM induced modest changes in the light-scattering fingerprints (most notably at the longer wavelengths) compared with corresponding fingerprints from control animals (FIG. 4A). This finding is consistent with the minimal carcinogenic effect of AOM in the proximal colon, which is supported by existing data. However, in the distal colon, the AOM-induced alterations of the fingerprints were much more dramatic (FIG. 4D vs. 4C), paralleling the increased carcinogenic efficacy in this region of the colon. We noted that the time point for which the alteration of light-scattering fingerprints was detected (i.e., 2 weeks after treatment with AOM) preceded the formation of ACF or other previously described conventional biomarkers.

III. Spectral Analysis

The light-scattering spectra Δl(λ) were used to obtain information about the size distribution of submicron intraepithelial structures in the size range from 40 to 800 nm (i.e., from macromolecular complexes to organelles). Representative size distribution curves were obtained from distal colon tissue sites of control and AOM-treated animals at 2, 5, 12, and 20 weeks after the carcinogen treatment, respectively. As carcinogenesis progressed, a variety of parameters (i.e., mean size, probable size, and relative proportion of larger structures) indicated an increase in particle dimensions. These findings are indicative of profound changes in the cellular nanoscale organization at an early stage of neoplastic transformation. Such alteration of cell nanoarchitecture has not been previously reported, most likely due to methodological limitations. Thus, 4D-ELF detection of microarchitectural changes in situ represents a major technological advance with potentially important biological and clinical ramifications.

IV. Spectral Slope

Spectral behavior of Δl(λ) depends on the size distribution of scattering structures. Generally, Δl(λ) is a declining function of wavelength and its steepness is related to the relative portion of structures of different sizes. Typically, larger structures tend to reduce the steepness of the decline of Δl(λ), whereas smaller scatterers tend to make Δl(λ) decrease with steeper wavelength. To analyze the data and characterize the spectral variations of Δl(λ), we obtained linear fits to Δl(λ) using linear regression analysis. The absolute value of the linear coefficient of the fit (in all measurements, the linear coefficient is negative due to the decrease of Δl with wavelength), which is referred hereafter to as the spectral slope, quantifies the dependence of the scattering spectrum on wavelength and may serve as an easily measurable marker to characterize the distribution of structures within the cells.

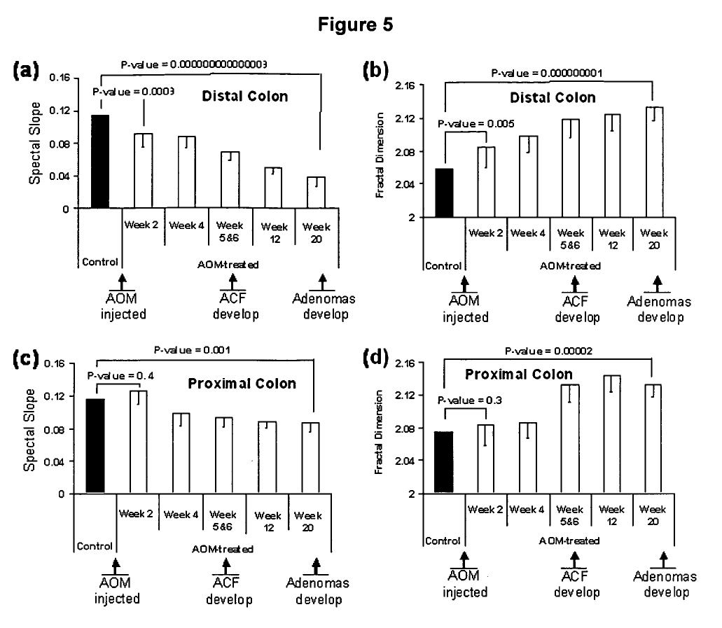

FIGS. 5B and C show alterations of the spectral slope in the AOM-treated rats compared with its control values. In the proximal colon, treatment with AOM failed to induce changes in the spectral slope at 2 weeks after the carcinogen treatment (P=0.43). This finding is consistent with only minimal carcinogenic effect of AOM in the proximal colon. On the contrary, in the distal colon, the spectral slope is dramatically decreased as early as 2 weeks after carcinogen treatment (P=0.0003) and continued to decrease over the course of the experiment (P<0.0001). Such progressive and highly statistically significant alteration of the spectral slope indicates that this parameter can be used as a marker for early precancerous transformations and its change is not due to the acute action of AOM.

V. Fractal Dimension

The angular distributions of the scattered light were used to calculate the fractal dimensions of the tissue microarchitecture. The angular distribution Δl(θ) at 550 nm for each tissue site was Fourier transformed to yield the 2-point mass density correlation function C(r)=(ρ[r]ρ[r′+r]), where ρ[r] is a local mass density at point r, which is proportional to the concentration of intracellular solids such as proteins, lipids, and DNA. C(r) quantifies the correlation between local tissue regions separated by distance r. For example, in a perfect solid, C(r) is a constant. On the other hand, for an object composed of randomly distributed material, C(r) vanishes rapidly with distance. At all tissue sites, C(r) was found to closely followed a power-law for several decades of r ranging from ˜1 to 50 μm. Such power-law density correlation functions have been extensively studied and are characteristic of a fractal-like or statistically self-similar organization. The general form of such C(r) is r^{D-3}, where D is referred to as fractal dimension. D was obtained from the linear slopes of C(r) in the linear regions of log-log scale.

As shown in FIG. 5B, in the distal colon fractal dimension was noted to be elevated as early as week 2 (P=0.005) and continued to markedly increase over time (P<0.0001). On the other hand, in the proximal colon, treatment with AOM failed to induce statistically significant alterations in fractal dimension. However, fractal dimension increased at later time points, albeit more modestly than that noted in the distal colon (FIG. 5D).

VI. PCA

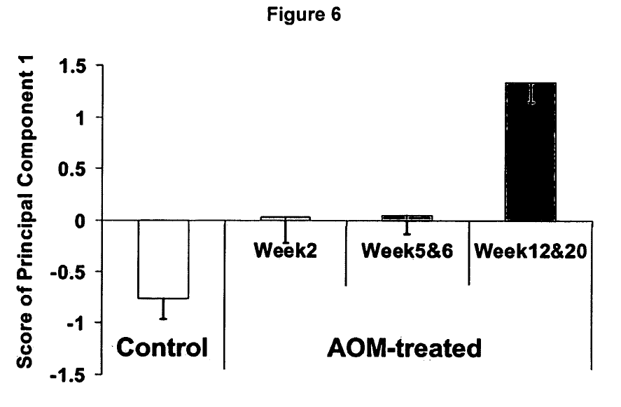

PCA was performed, and first the principal component of interest was determined. Typically in PCA, the first few principal components are responsible for most of the signal variations and the significance of higher-order principal components diminishes. In this data, principal component 1 (PC1) accounted for ˜99.3% of the data variance. Thus, PC1 is a convenient means to characterize the light scattering fingerprint data. As shown in FIG. 6, PC1 was significantly increased at 2 weeks in the distal colon (P=2×10⁻¹²) and this progressively continued over the course of the experiment (P=5×10⁻⁴³). On the other hand, PC1 was minimally elevated in the proximal colon (data not shown).

VII. Intersegment Variability

In the protocol used, each colonic segment had at least 4 distinct 1 mm² areas probed. To assess whether 4D-ELF could have a clinical role, it is of considerable importance to determine the number of measurements required to reliably detect premalignancy. Thresholds were established for categorizing an area as preneoplastic using PC1, linear slope, and fractal dimension. We analyzed sensitivity and specificity by applying these criteria to AOM- and saline-treated animals, respectively. Using this set of parameters, even at the earliest time point (2 weeks after injection of AOM), 90% of areas probed in the distal colon would correctly classify the animal as being exposed to carcinogen. This improved to 100% as the effects of the carcinogen progressed (weeks 12 and beyond). The specificity for all time points was 100%. This suggests that even at the earliest stages of colon carcinogenesis (2 weeks after treatment with AOM), 4 readings per colonic segment would provide a 99.99% probability of correctly diagnosing premalignancy. This accuracy far exceeds the capabilities of any conventional biomarker.

E. 4D-ELF Measurement of Blood Supply

Biomedical optics has frequently been used to measure tissue blood content by exploiting the characteristic absorption spectrum of hemoglobin in the visible range (light absorption at 542 and 577 nm wavelengths). Thus because no other molecules in biological tissue have similar absorption spectra, this provides a unique “spectral fingerprint” allowing remarkably accurate quantitation of RBCs.

4D-ELF enables us to accurately quantitate RBCs in both the subepithelial and mucosa/submucosa compartments, which is achieved via polarization gating. The differential polarization signal Δl(λ)=l_{∥}(λ)−l_{⊥}(λ) is primarily generated by scatterers located close to the tissue surface (up to ˜50 mm); that is, predominantly epithelial cells and the surrounding stroma with mucosal capillary plexus. On the other hand, l_{⊥}(λ) contains information about deeper tissues, up to ˜1 mm below the surface.

Blood content in superficial tissue (for example, pericryptal capillary plexus) was estimated by spectral analysis of Δl(λ). Firstly, we obtained the scattering maps, Δl_{RBC}(λ), of RBCs. Because Δl(λ)=Δl_{S}(λ)+α/Ω×Δl_{RBC}(λ), where Δl_{S}(λ) is the signal contributed by non-RBC components of superficial tissue, Ω represents a calibration constant, and RBC concentration in the superficial mucosa was obtained as the value of α that minimizes the hemoglobin absorption bands in Δl_{S}(A). Mucosal and superficial submucosal blood supply was assessed via l_{⊥}(λ) using a previously reported and well tested algorithm based on the diffusion approximation. In each animal, 4D-ELF blood supply measurements were taken from >100 tissue sites (˜1 mm² each) uniformly distributed throughout the colonic surface.

I. AOM Treated Rat Studies

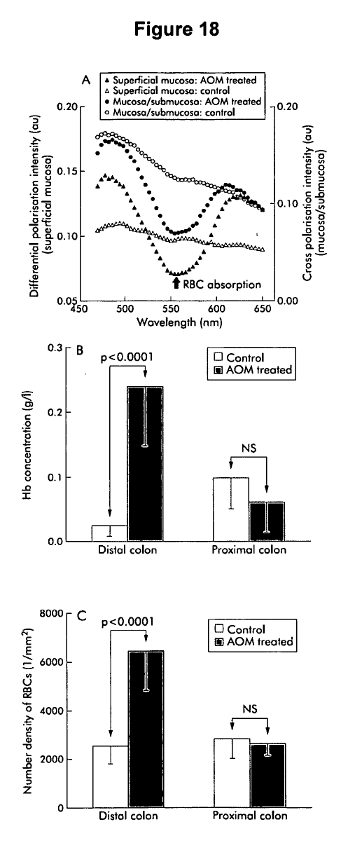

FIG. 18A shows representative spectra obtained from colons of AOM treated animals or age matched saline treated controls (two weeks post second injection). As shown, the spectra obtained from AOM treated animals showed the signatures of RBC increased absorption. Analysis of the spectra revealed a highly significant increase in distal colonic mucosal/submucosal blood content (p value<0.001; FIG. 18B). On the other hand, in the proximal colon, where the carcinogenic effects are generally minimal, no such increase was noted (FIG. 18B). When only the superficial (for example, pericryptal capillary plexus) component of blood content was assessed (FIG. 18C), a very similar picture emerged with a significant increase in the concentration of RBC in the distal (p<0.001) but not the proximal (p=0.3) colon. Thus EIBS preceded the development of ACF or adenomas, the classical early markers of colon carcinogenesis.

In our longitudinal studies, we observed a highly significant increase in the blood supply in the distal colon over time (ANOVA; p value<0.0001; FIG. 19B). In comparison, the proximal colon showed much less dramatic increase than the distal colon (p=0.12; FIG. 19C). EIBS therefore mirrors both the temporal (increase over time) as well as the spatial (distal dominance over proximal) progression of carcinogenesis. We also observed that the superficial blood content continued to be elevated over age matched saline treated controls (p<0.0001) (data not shown). Furthermore, in age matched controls, there was no significant increase in blood content over time (FIG. 19B, C).

II. MIN Mouse Studies

In order to demonstrate that EIBS is not model specific, we assessed blood content in the preneoplastic intestinal mucosa of the MIN mouse, another major model of experimental colon carcinogenesis. In this model, there is a germine mutation in the APC tumor suppressor gene, replicating the initiating genetic event in most human sporadic colon carcinogenesis. This leads to spontaneous and progressive development of intestinal adenomas. However, typically about 90% of the adenomas are located in the small bowel with the colon being minimally involved. We analyzed animals that were six weeks old, an age which precedes the occurrence of frank adenomatous polyps, thus being comparable with the premalignant stage (that is, two weeks post carcinogen) in our AOM model.

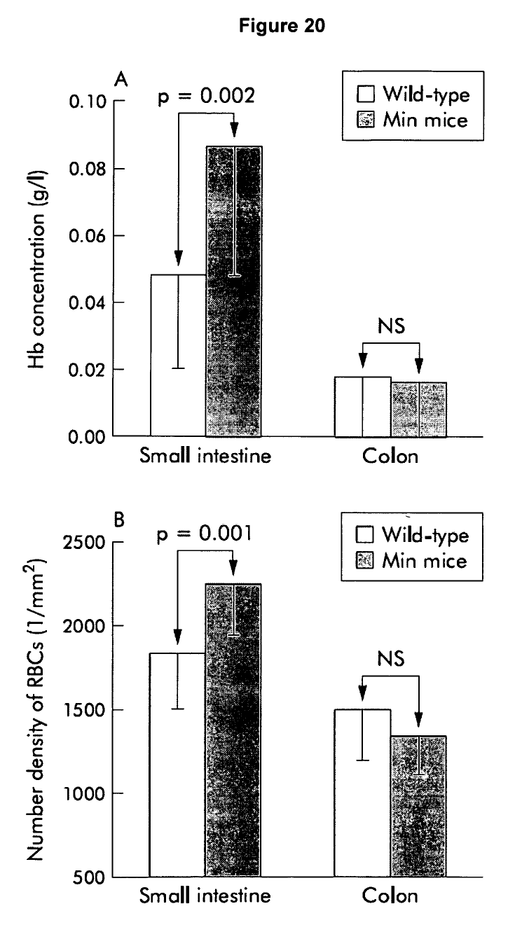

In the mice experiments, we used 16 male C57/BL6 mice with either adenomatous polyposis coli (APC) truncations at codon 850 (APC^{min}) or controls (wild-type APC gene) (Jackson Laboratory, Bar Harbor, Me., USA). Mice were killed at six weeks of age, the small bowel and colon isolated and opened longitudinally, and subjected to 4D-ELF to assess blood content. We noted a statistically significant increase in microvascular blood content in the small bowel but not in the colon, paralleling the location of future tumors (FIG. 20A). The superficial blood supply was also significantly increased compared with age matched wild-type mice in the small bowel but not in the colon (FIG. 20B).

III. Human Studies

Studies were conducted in accordance with the institutional review board of Evanston-Northwestern Healthcare. Two biopsies from endoscopically normal mid transverse colons were obtained from 37 patients undergoing screening colonoscopy. Patients were excluded if they had a history or endoscopic evidence of colitis or if the biopsy samples were too small for reliable estimation of blood content. Freshly harvested biopsies (within one hour) were subjected to 4D-ELF analysis.

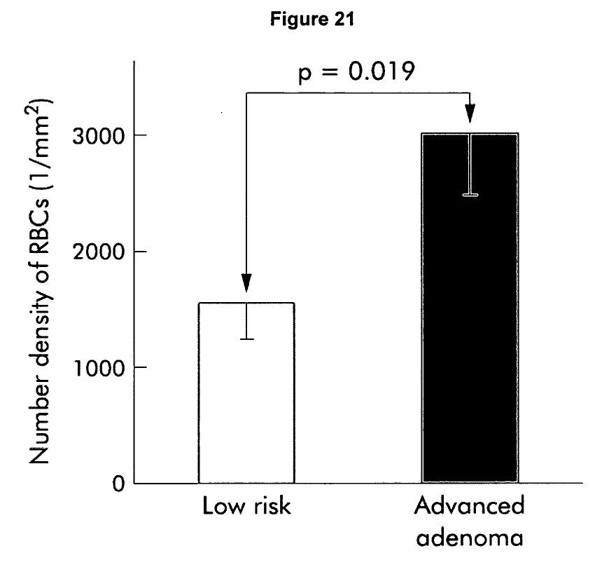

We compared the blood content from endoscopically normal mid trans colonic mucosa from patients with advanced adenomas (adenoma≧1 cm, high grade dysplasia or >25% villus component) versus those deemed to be at low risk for CRC (no history or present evidence of adenomas, colitis, or family history of CRC). There were no significant differences in age or sex between the low risk group and those that harbored advanced neoplasia. Importantly, none of the adenomas were located in the transverse colon (all lesions were located in the rectum, sigmoid colon, or caecum). Our data (FIG. 21) demonstrated marked augmentation of the blood content in the uninvolved (endoscopically normal) colonic mucosa in patients who harbored advanced neoplasia compared with those who were neoplasia free. Indeed, while our patient numbers were modest, this ˜3-fold increase was highly statistically significant (p<0.001). Limitations related to small biopsy size precluded accurate assessment of deeper blood content. While analysis of blood content in the distal colon would be most relevant to screening, the erythema/oedema associated with the phosphate based bowel preparatory regimen confounded blood content measurements in the rectum.

IV. Non-Optics Corroboration of EIBS

Immunoblot analysis of distal colonic mucosal scrapings was used as an additional methodology to assess hemoglobin content. One clear band at the appropriate molecular weight was noted (68 kDa) which was absent in negative controls (including lysates of two colon cancer cell lines HT-29 and HCT-116 and rat samples probed with secondary antibody alone; data not shown). At week 8 there was a marked increase in hemoglobin (142.4 (16.2)% of control, p=0.01). While the magnitude of EIBS determined immunoblot analysis was considerably less than noted with 4D-ELF, these data provide important non-optics corroboration of the EIBS phenomenon.

E. Characterization of Smooth Muscle Cell Proliferation

Human aortic smooth muscle cells (HASMCs) (Clonetics Inc.) were grown to confluence on either 25 μg/ml laminin coated or 25 μg/ml fibronectin (Sigma Inc.) coated glass coverslips. As previously discussed these protein substrates stimulate the cells to shift into the differentiated/contractile and proliferative/synthetic phenotypes respectively. Cells were grown in smooth muscle basal media (Clonetics Inc.) at 37° C., 95% relative humidity and 5% CO₂ for 5 to 8 days until they reached 80-90% confluence.

SMC differentiation status was confirmed with immunohistochemistry using specific phenotypic markers. Specifically, the contractile phenotype was confirmed by the presence of abundant smooth muscle α-actin, smooth muscle myosin heavy chain, and a low rate of proliferation. In contrast the proliferative phenotype was confirmed by the absence or decreased expression of smooth muscle α-actin, smooth muscle myosin heavy chain, and a high rate of proliferation.

I. Elastic Light-Scattering Fingerprints

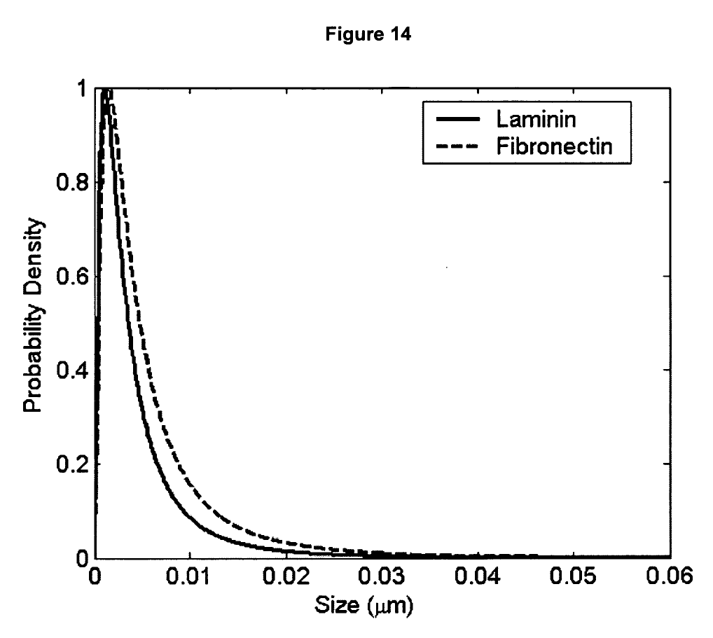

We focused on the analysis of the light scattering fingerprint data in two dimensions: wavelength and scattering angle. FIG. 14 shows representative size distributions obtained from SMCs grown on laminin and fibronectin substrates, respectively. Evidently, with the different effect of the extracellular matrix on the SMCs growth, the size distributions of SMCs grown on fibronectin shift towards larger sizes and its relative portion of larger structures, the mean, and the most probable sizes all become larger. These results were supported by transmission electron microscopy (TEM). Specifically, TEM studies have shown that the fibronectin promotes the transition of SMCs from a differentiated/contractile to a proliferative/synthetic phenotype, accompanying outgrowth of an extensive rough endoplasmic reticulum and a large Golgi complex. Endoplasmic reticulum is composed of tubules whose outer diameter ranges from 30 nm to 100 nm and the overall thickness of Golgi apparatus can range from 100 to 400 nm. The sizes of these two enlarged organelles confirmed by TEM fall into the range that the light scattering spectrum is sensitive to, from 40 nm to 800 nm. Four D-ELF results not only support previous findings, but also give more insight in the alteration of cell architecture at nanometer scale without destroying the live cells, which has not been achieved by conventional optical microscope or TEM.

II. Spectra Slope

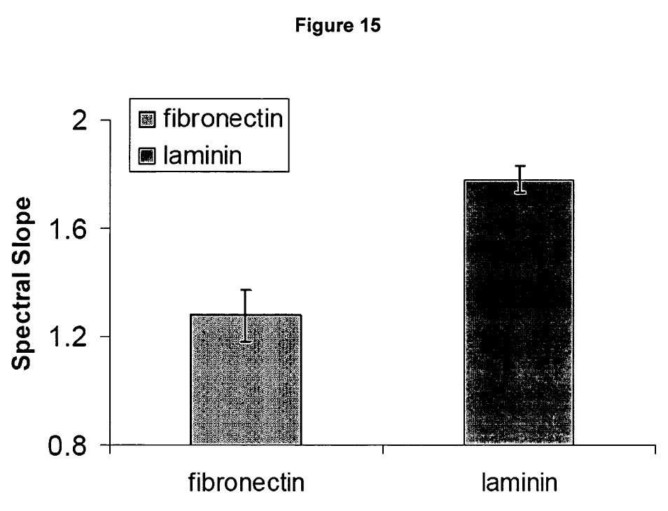

To analyze the data and characterize the spectral variations of l(λ), we obtained linear fits to log(l(λ)) vs. log(λ) using linear regression analysis. The absolute value of the linear coefficient of the fit (in all measurements the linear coefficient is negative due to the decrease of l(λ) with wavelength) (“spectral slope”) quantifies the dependence of the scattering spectrum on wavelength and may serve as an easily measurable marker to characterize the distribution of structures within the cells. As shown in FIG. 15, the values of spectral slopes obtained from the SMCs grown on fibronectin and laminin are significantly different. For the SMCs grown on fibronectin, the spectral slope is dramatically lower than one obtained for the SMCs grown on laminin (p-value=0.0000002), hence indicating larger sizes of intracellular structures of the fibronectin-grown SMCs. Such highly statistically significant alteration of the spectral slope indicates that this parameter may be used as a marker to monitor cellular structural changes.

III. PCA

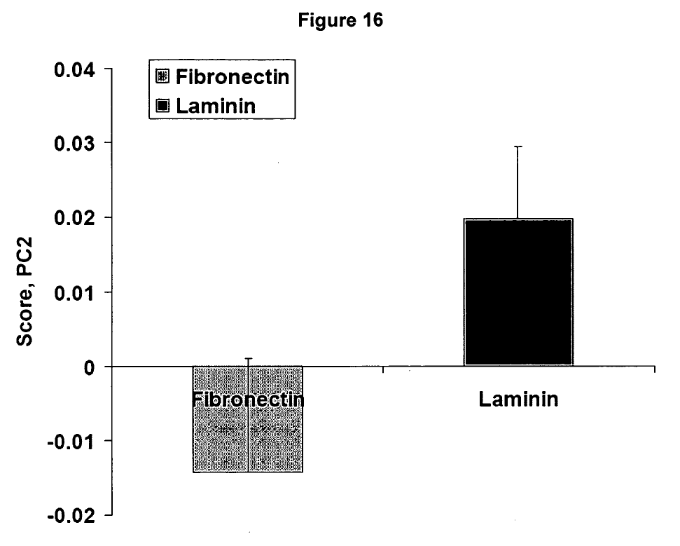

To further characterize the light scattering fingerprints differences, we performed PCA. We found that in our SMCs data, principal component 2 (PC2) accounts for the statistically significant portion of the whole data set. Therefore, PC2 may be used as a convenient measure to characterize the light scattering fingerprint data. FIG. 16 shows the change of score of PC2 in the SMCs grown on fibronectin and laminin with high statistical significance (p-value<0.0004).

F. Optical Characterization of Solid Polymeric Materials

This example is directed to the application of four-dimensional elastic light-scattering fingerprinting (4D-ELF) to the characterization of solid polymeric materials. Four D-ELF enables assessment of structural information in solid polymeric materials, which can be translated to information regarding mechanical properties. A key difference between 4D-ELF and traditional light scattering techniques (static and dynamic), is that the latter are limited to characterizing molecular weight and structure information of polymers in solution. Therefore, a benefit of 4D-ELF is that once a calibration curve is established, it can characterize mechanical properties and molecular weight information of crosslinked and solid phase linear polymers without subjecting the specimen to traditional destructive or perturbing tests that are often time consuming. Four D-ELF uses the angular and the spectral distribution of backscattered light from solid polymers to obtain structural information. The information may contain azimuthal and polarization dependence of backscattered light. Structural information at the nano- to micron scale can be obtained and converted to equivalent size information specific to the polymer of interest by fitting the computationally simulated spectra using Mie theory. The results obtained from 4D-ELF show a good correlation to the mechanical properties and molecular weight measured by traditional methods. Therefore, 4D-ELF is a fast, non-destructive, real-time, in-situ, and quantitative technique that will be a good addition to the arsenal of optical techniques that are currently used for polymer characterization. In particular, it could potentially be used as a quality control measure as it can monitor changes of polymer properties.

The application of 4D-ELF to structural characterization of solid polymeric materials was driven by the need to characterize, in a non-perturbing and real time manner, cross-linked elastomers originally developed for tissue engineering applications. However, the technique is applicable to other cross-linked materials and some linear polymers such as polystyrene as long as they are translucent. In particular, these examples describe the development of a novel family of citric acid-based biodegradable elastomers for tissue engineering and the present example teaches how to quickly and in a non-perturbing manner assess the extent of polymerization or cross-linking via intrinsic properties. These properties should be independent of specimen dimensions or sample processing and would have information regarding the ultrastructure of the material. A typical citric acid-based elastomer is poly(1,8 octanediol-co-citric acid) (POC). Another elastomer also under study is poly(glycerol sebacate) (PGS). Four D-ELF was used to characterize both POC and PGS elastomers as well as polystyrene of various molecular weights. As mechanical properties depend on the ultrastructure and chemical make up of a material, obtaining information pertinent to the degree of crosslinking (i.e. molecular weight between cross-links) should give insight into the mechanical properties of the material (i.e. Young's Modulus, tensile strength).

I. Four D-ELF Characterization of POC

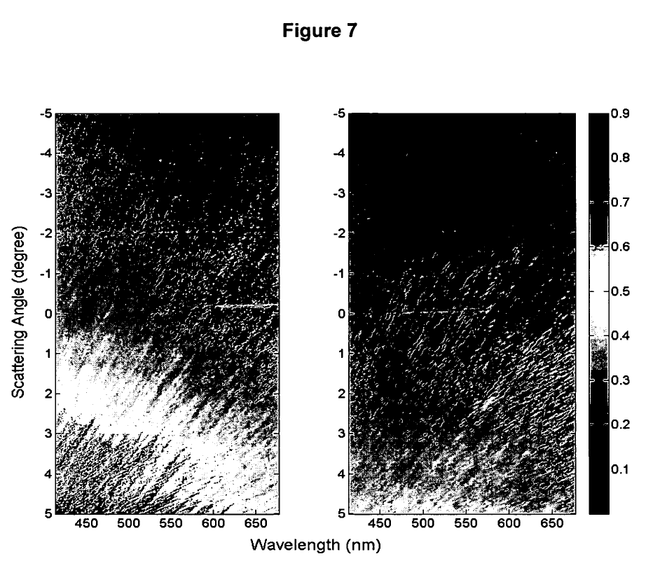

FIG. 7 shows the representative 4D-ELF recorded from POC films prepared under different conditions. The fingerprints show comprehensive 4 dimensional information: wavelength λ, scattering angle θ, azimuth of scattering φ, and polarization of scattered light from each measurement location of a polymer sample. The fingerprints are extremely sensitive to the changes of structure of a polymer. This unique characteristic makes 4D-ELF a good technique for fast, non-perturbing product identification and quality control methods.

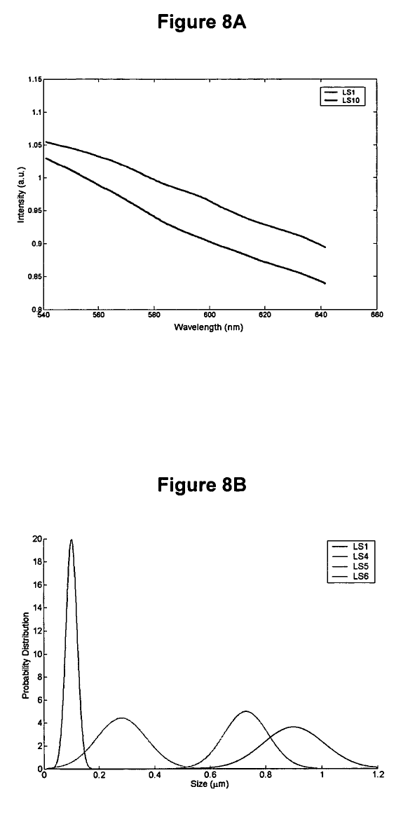

FIG. 8 shows the representative spectral distribution of the backscattered light (A) and the representative equivalent size distribution of POC obtained by fitting simulated spectra using Mie theory to polymer backscattered light spectra for each given scattering angle and azimuthal of scattering. POC synthesis under mild conditions (low temperature, low vacuum, short time) result in long polymer chains between crosslinks while POC synthesis under tough conditions (high temperature, high vacuum, long time) results in a highly crosslinked network (short polymer chains between crosslinks). Without being bound to any particular theory or mechanism of action, it may be that crosslinking creates scattering structures whose size decreases as the degree of crosslinking increases. The equivalent size distribution of scatteres within POC synthesized under these conditions ranges from 100 nm to 1 micron.

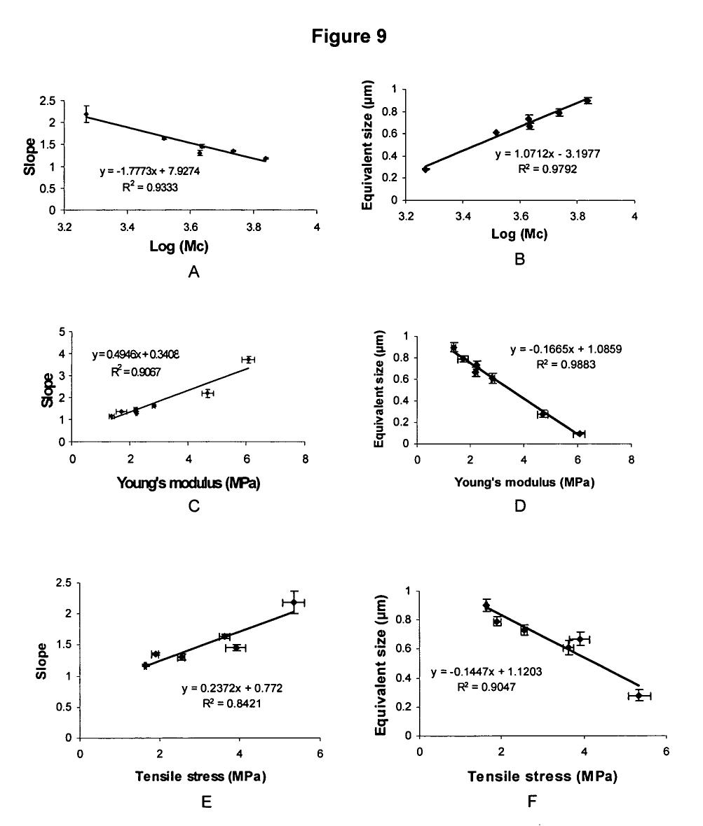

The slope fitted from the intensity versus wavelength spectra and equivalent sizes of polymer scatterers have strong linear correlations with logarithm molecular weight between crosslinks and mechanical properties (tensile stress and Young's modulus) of POC (FIG. 9). These results show that 4D-ELF can be used to characterize the molecular weight between crosslinks and mechanical properties once the standard curves are established for that material. This is a totally new light scattering method to characterize the molecular weight and mechanical properties of crosslinked polymers.

Swelling of a polymer sample is a traditional method for characterization of crosslinked polymers. According to Flory and Rehner's equilibrium swelling model, molecular weight between crosslinks can be calculated by Equation (1), which is different from the rubber elasticity theory method used by us to calculate Mc for POC and PGS (Tables 1 and 2). Using the swelling method, molecular weight between crosslinks can be calculated by Equation (1).

$$\frac{1}{{M}_{c}} = \frac{2}{{M}_{n}} - \frac{\frac{υ}{{V}_{1}} [ ln ( 1 - {υ}_{2 , s} ) + {υ}_{2 , s} + {χ}_{1} {υ}_{2 , s}^{2} ]}{{υ}_{2 , s}^{1 / 3} - \frac{{υ}_{2 , s}}{2}} \qquad ( 1 )$$

where Mc is the number average molecular weight of the linear polymer chain between cross-links, v is the specific volume of the polymer, V₁ is the molar volume of the swelling agent and χ₁ is the Flory-Huggins polymer-solvent interaction parameter. The equilibrium polymer volume fraction is v_{2,s}, which can be calculated from a series of weight measurements.

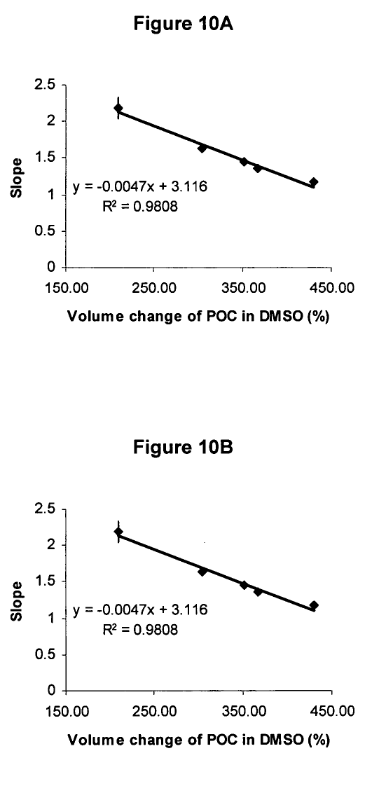

The equilibrium swelling volume of a crosslinked polymer network is an indicator of the molecular weight between crosslinks. Therefore, the spectral slope obtained by 4D-ELF measurements and equivalent scatterer size of POC (calculated via Mie Theory using a Gaussian distribution) were plotted against equilibrium swelling volumes of POC samples with increasing degree of crosslinking and revealed a substantially linear correlation (FIG. 10).

II. Four D-ELF Measurements for PGS Films

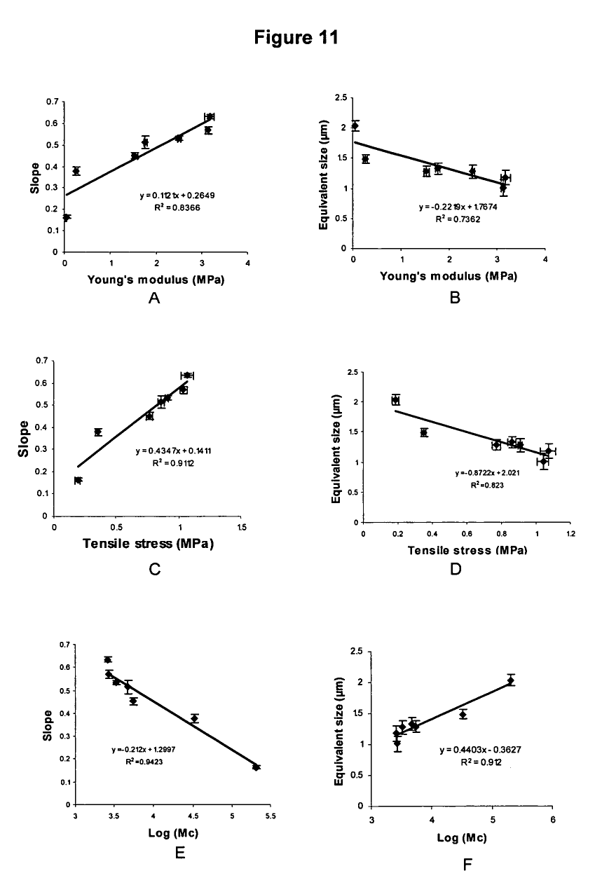

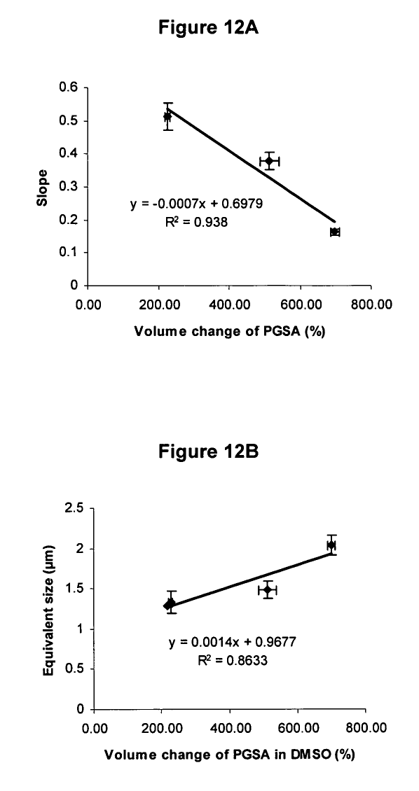

Four D-ELF measurements were also done on poly(glycerol sebacate) PGS, also a crosslinked elastomeric polymer, in order to test the applicability of this new method to other materials. The mechanical properties and molecular weight between crosslinks of PGS synthesized under different conditions were characterized and the results shown in Table 2. The spectral slope and equivalent size of polymers also show linear correlation with mechanical properties and molecular weight between crosslinks (FIG. 11). Volume changes of PGS by swelling study also show a good linear correlation with spectral slope and equivalent size of polymer obtained by 4D-ELF measurements (FIG. 12).

TABLE 1
Mechanical properties, the number of active network chain segment
per unit volume (crosslinking density: n) and molecular weight between
crosslinks (Mc) of POC synthesized under different conditions.

| | | Young's | Tensile Stress | n | Mc |
|---|---|---|---|---|---|
| POC | Polymerization condition | Modulus (MPa) | (MPa) | (mol/m³) | (g/mol) |
| LS1 | 80° C., no vacuum, 2 days | 1.38 ± 0.21 | 1.64 ± 0.05 | 182.59 ± 27.78 | 6874 ± 148 |
| LS2 | 80° C., high vacuum, 2 days | 1.72 ± 0.45 | 1.90 ± 0.22 | 227.58 ± 59.54 | 5445 ± 116 |
| LS3 | 120° C., high vacuum, 1 day | 2.84 ± 0.12 | 3.62 ± 0.32 | 375.77 ± 15.88 | 3301 ± 218 |
| LS4 | 120° C., high vacuum, 2 days | 3.13 ± 0.27 | 3.66 ± 0.61 | 414.14 ± 35.72 | 2971 ± 76 |
| LS5 | 120° C., high vacuum, 3 days | 4.69 ± 0.48 | 5.34 ± 0.66 | 620.68 ± 63.51 | 1857 ± 81 |
| LS6 | 140° C., high vacuum, 2 days | 6.07 ± 0.52 | 5.73 ± 1.39 | 803.14 ± 68.80 | 1516 ± 269 |
| LS7 | 80° C., no vacuum, 5 days | 2.21 ± 0.17 | 3.90 ± 0.60 | 292.41 ± 22.49 | 4326 ± 68 |
| LS8 | 80° C., no vacuum, 14 days | 2.24 ± 0.09 | 2.55 ± 0.21 | 296.38 ± 11.91 | 4265 ± 33 |

TABLE 2
Mechanical properties, the number of active network chain segment
per unit volume (crosslinking density: n) and molecular weight between
crosslinks (Mc) of PGS synthesized under different conditions.

| | Polymerization | Young's Modulus | Tensile Stress | n | Mc |
|---|---|---|---|---|---|
| PGSA | condition | (MPa) | (MPa) | (mol/m³) | (g/mol) |
| PA1 | Molar ratio 1/1, | 1.54 ± 0.18 | 0.77 ± 0.08 | 203.76 ± 23.82 | 5529 ± 57 |
| | 120° C., 3 days | | | | |
| PA2 | Molar ratio 1/1, | 2.50 ± 0.11 | 0.91 ± 0.04 | 330.78 ± 14.55 | 3324 ± 109 |
| | 120° C., 4 days | | | | |
| PA3 | Molar ratio 1/1.2, | 0.26 ± 0.13 | 0.35 ± 0.04 | 34.40 ± 17.20 | 32605 ± 1069 |
| | 120° C., 2 days | | | | |
| PA4 | Molar ratio 1/1.2, | 1.77 ± 0.11 | 0.86 ± 0.08 | 234.19 ± 14.55 | 4702 ± 269 |
| | 120° C., 3 days | | | | |
| PA5 | Molar ratio 1/1.2, | 3.17 ± 0.31 | 1.07 ± 0.05 | 419.43 ± 41.01 | 2630 ± 16 |
| | 120° C., 4 days | | | | |

III. Four D-ELF Measurements for Linear Ppolymer: Polystyrene

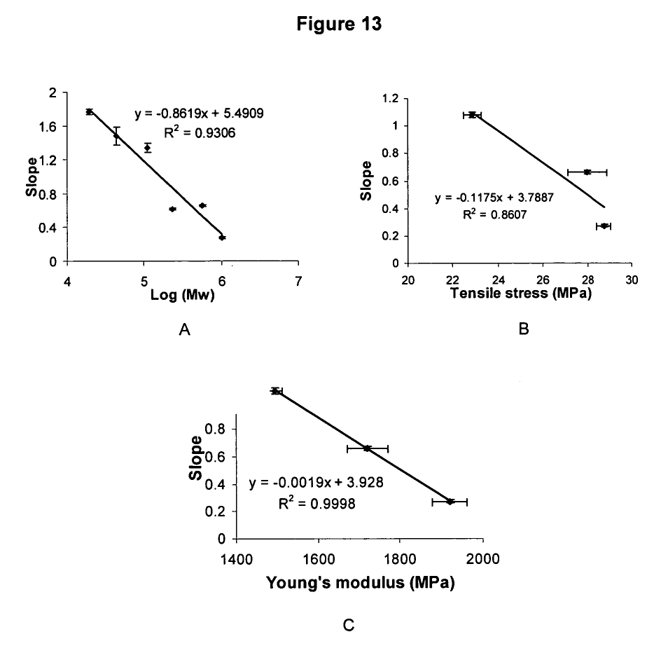

Four D-ELF can also be extended for characterization of linear solvent soluble polymers. Polystyrene standards were chosen as model linear polymers for 4D-ELF measurements since they are widely used as standards or models for molecular weight (gel permeation chromatography) and size distribution investigations. The results show that spectral slope has a strong linear correlation with molecular weight, tensile stress and Young's modulus of polystyrene standards (FIG. 13). Therefore, 4D-ELF is also well suitable for mechanical properties and molecular weight characterization of linear polymers.

### REFERENCES

Agrawal S. Importance of nucleotide sequence and chemical modifications of antisense oligonucleotides. Biochim Biophys Acta 1999; 1489: 53-68.
Ahnen D J, Byers T. Proliferation happens. JAMA 1998;280:1095-6.
Akagi K, Ikeda Y, Sumiyoshi Y, et al. Estimation of angiogenesis with anti-CD105 immunostaining in the process of colorectal cancer development. Surgery 2002; 131:S109-13.
Akpalu, Y. A., Y. Lin. Journal of Polymer Science: Part B: Polymer Physics 2002; 40: 2714.
Allcock, F. W. L. Harry R, e. New Jersey: Prentice-Hall, Inc.1990.
Ameer, G., Yang J., Webb A. US Published Patent Application Publication No. US 2005/0063939 (2005).
American Heart Association, 2002 Heart and stroke statistical update. Dallas, Tex., 2001.
Anti M, Marra G, Armelao F, et al. Rectal epithelial cell proliferation patterns as predictors of adenomatous colorectal polyp recurrence. Gut 1993;34:525-30.
Aotake T, Lu C D, Chiba Y, et al. Changes of angiogenesis and tumor cell apoptosis during colorectal carcinogenesis. Clin Cancer Res 1999;5:135-42.
Backman V, Gopal V, Kalashnikov M, Badizadegan K, Gurjar R, Wax A, Georgakoudi I, Mueller M, Boone C W, Dasari R R, Feld M S. Measuring cellular structure at submicrometer scale with light scattering spectroscopy. IEEE J Sel Top Quantum Electron 2001; 7:887-893.
Backman V, Gurjar R, Badizadegan K, Itzkan L, Dasari R R, Perelman L T, Feld M S. Polarized light scattering spectroscopy for quantitative measurement of epithelial cellular structures in situ. IEEE J Sel Top Quantum Electron 1999;5:1019-1026.
Backman V, Wallace M B, Perelman L T, Arendt J T, Gurjar R, Muller M G, Zhang Q, Zonios G, Kline E, McGillican T, Shapshay S, Valdez T, Badizadegan K, Crawford J M, Fitzmaurice M, Kabani S, Levin H S, Seiler M, Dasari R R, Itzkan I, Van Dam J, Feld M S. Detection of preinvasive cancer cells. Nature 2000;406:35-36.
Baish J W, Jain R K. Fractals and cancer. Cancer Res 2000;60:3683-8.
Banerjee A, Quirke P. Experimental models of colorectal cancer. Dis Colon Rectum 1998;41 :490-505.
Barlett, M., H. Jiang. Physical Review E. Statistical Physics, Plasmas, Fluids, and Related Interdisciplinary Topics 2002; 65: 031906/1.
Barnes C J, Lee M, Hardman W E, et al. Aspirin, age, and proximity to lymphoid nodules influence cell proliferation parameters in rat colonic crypts. Cell Prolif 1995;28:59-71.
Barth, H. G., e. New York: Wiley Interscience.1984.
Batlle E, Sancho E, Franci C, et al. The transcription factor snail is a repressor of E-cadherin gene expression in epithelial tumor cells. Nat Cell Biol 2000;2:84-9.
Bernstein C, Bernstein H, Garewal H, Dinning P, Jabi R, Sampliner R E, McCuskey M K, Panda M, Roe D J, L'Heureux L, Payne C. A bile acid-induced apoptosis assay for colon cancer risk and associated quality control studies. Cancer Res 1999;59:2353-2357.
Bienz M, Clevers H. Linking colorectal cancer to Wnt signaling. Cell 2000; 103:311-20.
Bigio I J, Mourant J R. Ultraviolet and visible spectroscopies for tissue diagnostics: fluorescence spectroscopy and elastic-scattering spectroscopy. Phys Med Biol 1997;42:803-814.
Bigio, I. J., S. G. Bown, G. Briggs, C. Kelley, S. Lakhani, D. Pickard, P. M. Ripley, I. G. Rose, and C. Saunders, “Diagnosis of breast cancer using elastic-scattering spectroscopy: Preliminary clinical results,” J. Biomed. Opt., vol. 5, pp. 221-228, 2000.
Bird R P. Role of aberrant crypt foci in understanding the pathogenesis of colon cancer. Cancer Lett 1995;93:55-71.
Blanco M J, Moreno-Bueno G, Sarrio D, et al. Correlation of Snail expression with histological grade and lymph node status in breast carcinomas. Oncogene 2002;21 :3241-6.
Bordenave L, Remy-Zolghadri M, Fernandez P, Bareille R, Midy D. Endothelium 1999;6:267-275.
Borse G J. Fortran 77 and numerical methods for engineers. Boston, Mass.: PWS-KENT, 1991.
Bos G W, Poot A A, Beugeling T, van Aken W G, Feijen I. Archieve of Physiology and Biochemistry 1998; 106: 100-115.
Braakhuis B, Tabor M, Kummer J, Leemans C, Brakenhoff R. A genetic explanation of slaughter's concept of field cancerization: evidence and clinical implications. Cancer Res 2003;63:1727-1730.
Carnagey I, Hem-Anderson D, Ranieri I, Schmidt C E. I. Biomed. Mater. Res. Part B: Appl Biomater 2003;65B:171-179.
Cerussi, A. E., D. Jakubowski, N. Shah, F. Bevilacqua, R. Lanning, A. J. Berger, D. Hsiang, J. Butler, R. F. Holcombe, and B. J. Tromberg, “Spectroscopy enhances the information content of optical mammography,” J. Biomed. Opt., vol. 7, pp. 60-71, 2002.
Chan T A. Nonsteroidal anti-inflammatory drugs, apoptosis, and coloncancer chemoprevention. Lancet Oncol 2002;3:166-74.
Chen L C, Hao C Y, Chiu Y S, et al. Alteration of gene expression in normalappearing colon mucosa of APC(min) mice and human cancer patients. Cancer Res 2004;64:3694-700.
Chiba M, Komatsu K. I. Biomech. 1993;26:561-570.
Chu, B., e. 2nd ed. New York: Academic Press.1991.
Chung, D. C., “The genetic basis of colorectal cancer: insights into critical pathways of tumorigenesis,” Gastroenterol., vol. 119, pp. 854-865, 2000.
Coffey D S. Self-organization, complexity and chaos: the new biology for medicine. Nat Med 1998;4:882-5.
Consigny P M. Long Term Eff Med Implants 2000;10:79-95.
Cross S S. Fractals in pathology. J Pathol 1997;182:1-8.
Cruz-Correa M, Cui H, Giardiello F M, et al. Loss of imprinting of insulin growth factor II gene: a potential heritable biomarker for colon neoplasia predisposition. Gastroenterology 2004; 126:964-70.
De Vita F, Orditura M, Lieto E, et al. Elevated perioperative serum vascular endothelial growth factor levels in patients with colon carcinoma. Cancer 2004; 100:270-8.
Dekker A, Reitsma K. Beugeling T, Feijen I, van Aken W G. Biomaterials 1991;12:130-138.
Demos S G, Alfano R R. Optical polarization imaging. Appl Optics 1997;36:150-155.
Dillon W R, Goldstein M. Multivariate analysis: methods and applications. New York: Wiley, 1984.
Drezek R, Guillaud M, Collier T, Boiko I, Malpica A, Macaulay C, Follen M, Richards-Kortum R. Light scattering from cervical cells throughout neoplastic progression: influence of nuclear morphology, DNA content, and chromatin texture. J Biomed Opt 2003;8:7-16.
Easwaran V, Lee S H, Inge L, et al. Beta-catenin regulates vascular endothelial growth factor expression in colon cancer. Cancer Res 2003;63:3145-53.
Edelstein P S, Thompson S M, Davies R J. Altered intracellular calcium regulation in human colorectal cancers and in “normal” adjacent mucosa. Cancer Res 1991;51:4492-4.
Folkman J, Watson K, Ingber D, et al. Induction of angiogenesis during the transition from hyperplasia to neoplasia. Nature 1989;339:58-61.
Fujita N, Jaye D L, Kajita M, Geigerman C, Moreno C S, Wade P A. MT A3, a Mi-2/NuRD complex subunit, regulates an invasive growth pathway in breast cancer. Cell 2003; 113:207-19.
Gedde, U. W., e. 1st ed. New York: Chapman & Hall.1995.
Georgakoudi I, Jacobson B C, Muller M G, et al. NAD(P)H and collagen as in vivo quantitative fluorescent biomarkers of epithelial precancerous changes. Cancer Res 2002;62:682-7.
Georgakoudi I, Jacobson B C, Van Dam J, Backman V, Wallace M B, Muller M G, Zhang Q, Badizadegan K, Sun D, Thomas G A, Perelman L T, Feld M S. Fluorescence, reflectance, and light-scattering spectroscopy for evaluating dysplasia in patients with Barrett's esophagus. Gastroenterology 2001; 120:1620-1629.
Georgakoudi I, Motz J, Backman V, et al. Quantitative characterization of biological tissue using optical spectroscopy. In: Vo-Dinh T, ed. Biomedical photonics handbook. New York: CRC Press, 2003.
Georgakoudi I, Sheets E E, Muller M G, et al. Trimodal spectroscopy for the detection and characterization of cervical precancers in vivo. Am J Obstet Gynecol 2002;186:374-82.
Gerdes H, Gillin J S, Zimbalist E, Urmacher C, Lipkin M, Winawer S J. Expansion of the epithelial cell proliferative compartment and frequency of adenomatous polyps in the colon correlate with the strength of family history of colorectal cancer. Cancer Res 1993;53:279-82.
Giardiello F M, Hamilton S R, Hylind L M, Yang V W, Tamez P, Casero R A Jr. Ornithine decarboxylase and polyamines in familial adenomatous polyposis. Cancer Res 1997;57:199-201.
Gouesbet, G., G. Grehan,e. New York: Plenum Press.1988.
Grady W M. Genetic testing for high-risk colon cancer patients. Gastroenterology 2003; 124:1574-94.
Greenwald S E, Berry C L. I. Pathol. 2000;190:292-299.
Greisler H P, Chatelier R C, Gengenbach T R, Johnson G, Steele I G. Biomater Sci Polymer Ed. 1994;5 :531-554.
Griffith L G. Polymeric biomaterials. Acta Mater. 2000,48:263-277
Grille S J, Bellacosa A, Upson J, et al. The protein kinase Akt induces epithelial mesenchymal transition and promotes enhanced motility and invasiveness of squamous cell carcinoma lines. Cancer Res 2003;63: 2172-8.
Groos S, Reale E, Luciano L. General suitability of techniques for in situ detection of apoptosis in small intestinal epithelium. Anat Rec 2003;272A:503-13.
Guldberg R E. Consideration of mechanical factors. Ann. N. Y. Acad. Sci. 2002;961:312-314.
Gurjar R S, Backman V, Perelman L T, Georgakoudi I, Badizadegan K, Itzkan I, Dasari R R, Feld M S. Imaging human epithelial properties with polarized light-scattering spectroscopy. Nat Med 2001;7:1245-1248.
Hemavathy K, Ashraf S I, Ip Y T. Snail/Slug family of repressors: slowly going into the fast lane of development and cancer. Gene 2000;257: 1-12.
Hoffman J A, Giraudo E, Singh M, et al. Progressive vascular changes in a transgenic mouse model of squamous cell carcinoma. Cancer Cell 2003;4:383-91.
Hoglund M, Gisselsson D, Hansen G B, Sall T, Mitelman F, Nilbert M. Dissecting karyotypic patterns in colorectal tumors: two distinct but overlapping pathways in the adenoma-carcinoma transition. Cancer Res 2002;62:5939-5954.
Hoglund M, Gisselsson D, Sall T, Mitelman F. Coping with complexity: multivariate analysis of tumor karyotypes. Cancer Genet Cytogenet 2002; 135:103-109.
Horwitz J, Cohen E, Goldberg M, et al. Micro-architectural alterations in endoscopically-normal mucosa provides accurate risk stratification for colorectal neoplasia. Am J Gastroenterol 2004;99:A11.
Hubbell I A, Massia S P, Desai N P, Drumheller P D. Biotechnology 1991;9:568-572.
Hurwitz H, Fehrenbacher L, Novotny W, et al. Bevacizumab plus irinotecan, fluorouracil, and leucovorin for metastatic colorectal cancer. N Engl J Med 2004;350:2335-42.
Iacopetta B. Are there two sides to colorectal cancer? Int J Cancer 2002;101 :403-408.
Ishimaru A. Wave propagation and scattering in random media. Piscataway: Wiley IEEE Computer Society Press, 1999.
Issa J P, Vertino P M, Wu J, et al. Increased cytosine DNA-methyltransferase activity during colon cancer progression. J Natl Cancer Inst 1993;85:1235-40.
Iversen P L. Phosphorodiamidate morpholino oligomers: favorable properties for sequence-specific gene inactivation. Curr Opin Mol Ther 2001;3:235-8.
Jackson P E, O'Connor P J, Cooper D P, Margison G P, Povey A C. Associations between tissue-specific DNA alkylation, DNA repair and cell proliferation in the colon and colon tumour yield in mice treated with 1,2-dimethylhydrazine. Carcinogenesis 2003;24:527-533.
Jacoby R F, Seibert K, Cole C E, Kelloff G, Lubet R A. The cyclooxygenase-2 inhibitor celecoxib is a potent preventive and therapeutic agent in the min mouse model of adenomatous polyposis. Cancer Res 2000;60:5040-4.
Jacques S L, Roman J R, Lee K. Imaging superficial tissues with polarized light. Lasers Surg Med 2000;26:119-129.
Jacques, S. L., J. C. Ramella-Roman, and K. Lee, “Imaging skin pathology with polarized light,” Journal of Biomedical Optics, vol. 7, pp. 329-340, 2002.
Jaiswal A S, Narayan S. Upstream stimulating factor-1 (USF1) and USF2 bind to and activate the promoter of the adenomatous polyposis coli (APC) tumor suppressor gene. J Cell Biochem 2001 ;81 :262-77.
Jass J R, Whitehall V L J, Young J, Leggett B A. Emerging concepts in colorectal neoplasia. Gastroenterology 2002; 123:862-876.
Jemal A, Murray T, Samuels A, Ghafoor A, Ward E, Thun M J. Cancer statistics, 2003. CA Cancer J Clin 2003;53:5-26.
Jiao W, Miyazaki K, Kitajima Y. Inverse correlation between E-cadherin and Snail expression in hepatocellular carcinoma cell lines in vitro and in vivo. Br J Cancer 2002;86:98-101.
Joyce J A, Laakkonen P, Bernasconi M, et al. Stage-specific vascular markers revealed by phage display in a mouse model of pancreatic islet tumorigenesis. Cancer Cell 2003;4:393-403.
Kanazawa T, Watanabe T, Kazama S, Tada T, Koketsu S, Nagawa H. Poorly differentiated adenocarcinoma and mucinous carcinoma of the colon and rectum show higher rates of loss of heterozygosity and loss of E-cadherin expression due to methylation of promoter region. Int J Cancer 2002; 102:225-9.
Kettunen H L, Kettunen A S, Rautonen N E. Intestinal immune responses in wild-type and Apcmin/+mouse, a model for colon cancer. Cancer Res 2003; 63:5136-42.
Kim B S, Putnam A J, Kulik T I, Mooney D I. Biotechnol. Bioeng. 1998;57:46-54.
Kim Y, Liu Y, Wali R K, Roy H K, Goldberg M J, Kromine A K, Chen K, Backman V. Simultaneous measurement of angular and spectral properties of light scattering for characterization of tissue microarchitecture and its alteration in early precancer. IEEE J Sel Top Quantum Electron 2003;9:243-257.
Kishimoto Y, Morisawa T, Hosoda A, Shiota G, Kawasaki H, Hasegawa J. Molecular changes in the early stage of colon carcinogenesis in rats treated with azoxymethane. J Exp Clin Cancer Res 2002;21 :203-211.
Kobaek-Larsen, M., I. Thorup, A. Diederichsen, C. Fenger, and M. R. Hoitinga, “Review of colorectal cancer and its metastases in rodent models: Comparative aspects with those in humans,” Comput. Med., vol. 50, pp. 16-26, 2000.
Kweon H Y, Yoo M K, Park I K, Kim T H, Lee H C, Lee H S, Oh I S, Akaike T, Cho C S. Biomaterials 2003;24:801-808.
Langer R, Vacanti I P. Tissue engineering. Science 1993,260:920
Lee M C, Haut R C. I. Biomech. 1992;25 :925-927.
Lee S G, Rho H M. Transcriptional repression of the human p53 gene by hepatitis B viral X protein. Oncogene 2000;19:468-71.
Lewis J D, Ng K, Hung K E, Bilker W B, Berlin J A, Brensinger C, Rustgi A K. Detection of proximal adenomatous polyps with screening sigmoidoscopy—a systematic review and meta-analysis of screening colonoscopy. Arch Intern Med 2003;163:413-420.
Lieberman D A, Weiss D G, Bond J H, Ahnen D J, Garewal H, Chejfec G, Harford W V, Provenzale D, Sontag S, Schnell T, Campbell D R, Durbin T E, Nelson D B, Ewing S L, Triadafilopoulos G, Ramirez F C, Lee J G, Collins J F, Fennerty B, Johnston T K, Corless C T, McQuaid K R, Sampliner R E, Morales T G, Fass R, Smith R, Maheshwari Y. Use of colonoscopy to screen asymptomatic adults for colorectal cancer. Veterans Affairs Cooperative Study Group 380. N Engl J Med 2000;343:162-168.
Lin W C, Toms S A, Jansen E D, Mahadevan-Jansen A. Intraoperative application of optical spectroscopy in the presence of blood. IEEE J Sel Top Quantum Electron 2001;7:996-1003.
Lisowski, M. S., Q. Liu, J. Cho, J. Runt, F. Yeh, B. S. Hsiao. Macromolecules 2000; 33: 4842.
Lu Z, Ghosh S, Wang Z, Hunter T. Downregulation of caveolin-1 function by EGF leads to the loss of E-cadherin, increased transcriptional activity of h-catenin, and enhanced tumor cell invasion. Cancer Cell 2003; 4:499-515.
Lynch H T, Schuelke G S, Kimberling W J, et al. Hereditary nonpolyposis colorectal cancer (Lynch syndromes I and II). II. Biomarker studies. Cancer 1985;56:939-51.
Mahmoud N N, Bilinski R T, Churchill M R, Edelmann W, Kucherlapati R, Bertagnolli M M. Genotype-phenotype correlation in murine Apc mutation: differences in enterocyte migration and response to sulindac. Cancer Res 1999;59:353-9.
Martin C, Connelly A, Keku T O, Mountcastle S B, Galanko J, Woosley J T, Schliebe B, Lund P K, Sandler R S. Nonsteroidal antiinflammatory drugs, apoptosis, and colorectal adenomas. Gastroenterology 2002; 123:1770-1777.
McDonald D M, Choyke P L. Imaging of angiogenesis: from microscope to clinic. Nat Med 2003;9:713-25.
McGarrity T J, Peiffer L P. Protein-kinase-c activity as a potential marker for colorectal neoplasia. Dig Dis Sci 1994;39:458-463.
Mills S J, Shepherd N A, Hall P A, Hastings A, Mathers J C, Gunn A. Proliferative compartment deregulation in the non-neoplastic colonic epithelium of familial adenomatous polyposis. Gut 1995;36:391-4.
Misof K, Rapp G, Fmtzi P A. Biophys. I. 1997;72:1376-1381.
Miyoshi A, Kitajima Y, Sumi K, et al. Snail and SIP1 increase cancer invasion by upregulating MMP family in hepatocellular carcinoma cells. Br J Cancer 2004;90: 1265-73.
Montag A G, Bartels P H, Dytch H E, Lermapuertas E, Michelassi F, Bibbo M. Karyometric features in nuclei near colonic adenocarcinoma-statistical analysis. Anal QuantCytol Histol 1991;13: 159-167.
Moran A E, Carothers A M, Weyant M J, Redston M, Bertagnolli M M. Carnosol inhibits h-catenin tyrosine phosphorylation and prevents adenoma formation in the C57BL/6J/Min/+(Min/+) mouse. Cancer Res 2005;65: 1097-104.
Moran A E, Hunt D H, Javid S H, Redston M, Carothers A M, Bertagnolli M M. Apc deficiency is associated with increased Egfr activity in the intestinal enterocytes and adenomas of C57BLU6J-Min/+mice. J Biol Chem 2004; 279:43261-72.
Mori Y, Selaru F M, Sato F, Yin J, Simms L A, Xu Y, Olaru A, Deacu E, Wang S, Taylor J M, Young J, Leggett B, Jass J R, Abraham J M, Shibata D, Meltzer S J. The impact of microsatellite instability on the molecular phenotype of colorectal tumors. Cancer Res 2003; 63:4577-4582.
Mourant J R, Fuselier T, Boyer J, Johnson T M, Bigio I J. Predictions and measurements of scattering and absorption over broad wavelength ranges in tissue phantoms. Appl Optics 1997;36:949-957.
Mourant, J. R., I. J. Bigio, J. Boyer, R. L. Conn, T. Johnson, and T. Shimada, “Spectroscopic diagnosis of bladder cancer with elastic light scattering,” Laser Surg. Med., vol. 17, pp. 350-357, 1995.
Mourant, J. R., J. P. Freyer, A. H. Hielscher, A. A. Eick, D. Shen, and T. M. Johnson, “Mechanisms of light scattering from biological cells relevant to noninvasive optical-tissue diagnostics,” Appl. Opt., vol. 37, pp. 3586-3593, 1998.
Mourant, J. R., T. M. Johnson, S. Carpenter, A. Guerra, T. Aida, and J. P. Freyer, “Polarized angular dependent spectroscopy of epithelial cells and epithelial cell nuclei to determine the size scale of scattering structures,” J. Biomed. Opt., vol. 7, pp. 378-387, 2002.
Muller M G, Valdez T A, Georgakoudi I, et al. Spectroscopic detection and evaluation of morphologic and biochemical changes in early human oral carcinoma. Cancer 2003;97:1681-92.
Murff H J, Spigel D R, Syngal S. Does this patient have a family history of cancer? An evidence-based analysis of the accuracy of family cancer history. JAMA 2004;292:1480-9.
Nelson W J, Nusse R. Convergence of Wnt, h-catenin, and cadherin pathways. Science 2004;303:1483-7.
Odin E, Wettergren Y, Nilsson S, et al. Altered gene expression of folate enzymes in adjacent mucosa is associated with outcome of colorectal cancer patients. Clin Cancer Res 2003;9:6012-9.
Peppas, N. A., B. D. Barr, in Hydrogels in Medicine and Pharmacy, (Eds: N. A. Peppas). CRC Press: Boca Raton, Fla. 1988 p. 27.
Perelman L T, Backman V, Wallace M, Zonios G, Manoharan R, Nusrat A, Shields S, Seiler M, Lima C, Hamano T, ltzkan I, Van Dam J, Crawford J M, Feld M S. Observation of periodic fine structure in reflectance from biological tissue: a new technique for measuring nuclear size distribution. Phys Rev Lett 1998;80:627-630.
Perelman, L. T. and V. Backman, “Light Scattering Spectroscopy of Epithelial Tissues: Principals and Applications,” in Handbook of Optical Biomedical Diagnostics, V. V. Tuchin, Ed. Bellingham, Wash.: SPIE—The International Society for Optical Engineering, 2002, pp. 675-724.
Poser I, Dominguez D, de Herreros A G, Varnai A, Buettner R, Bosserhoff A K. Loss of E-cadherin expression in melanoma cells involves up-regulation of the transcriptional repressor Snail. J Biol Chem 2001; 276:24661-6.
Ramanujam N, Mitchell M F, Mahadevan A, Thomsen S, Malpica A, Wright T, Atkinson N, Richards-Kortum R. Development of a multivariate statistical algorithm to analyze human cervical tissue fluorescence spectra acquired in vivo. Lasers Surg Med 1996;19:46-62.
Rao C V, Indranie C, Simi B, et al. Chemopreventive properties of a selective inducible nitric oxide synthase inhibitor in colon carcinogenesis, administered alone or in combination with celecoxib, a selective cyclooxygenase-2 inhibitor. Cancer Res 2002;62:165-70.
Roncucci, L., M. Pedroni, F. Vaccina, P. Benatti, L. Marzona, and A. De Pol, “Aberrant crypt foci in colorectal carcinogenesis. Cell and crypt dynamics,” Cell Prolif., vol. 33, pp. 1-18, 2000.
Rosivatz E, Becker I, Specht K, et al. Differential expression of the epithelial-mesenchymal transition regulators snail, SIP1, and twist in gastric cancer. Am J Pathol 2002;161:1881-91.
Ross, R., “The pathogenesis of atherosclerosis—an update,” N. Engl. J. Med., vol. 314, pp. 488-500, 1986.
Roy H, Liu Y, Wali R K, Kim Y L, Kromine A K, Goldberg M J, and Backman V. “Four-Dimensional Elastic Light-Scattering Fingerprints as Preneoplasic Markers in the Rat Model of Colon Carcinogenesis”, GASTROENTEROLOGY 2004; 126:1071-1081.
Roy H K, Gulizia J, DiBaise J K, et al. Induction of epithelial apoptosis in polyethylene glycol-induced chemoprevention in APCmin mice. Cancer Lett. In press 2004.
Roy H K, Iversen P, Hart J, et al. Downregulation of SNAIL suppresses MIN mouse tumorigenesis: modulation of apoptosis, proliferation and fractal dimension. Mol Cancer Ther 2004;3:1159-65.
Roy H K, Karolski W J, Ratashak A. Distal bowel selectivity in the chemoprevention of experimental colon carcinogenesis by the non-steroidal anti-inflammatory drug nabumetone. Int J Cancer 2001 ;92:609-615.
Roy H K, Karoski W J, Ratashak A, et al. Chemoprevention of intestinal tumorigenesis by nabumetone: induction of apoptosis and Bcl-2 downregulation. Br J Cancer 2001;84:1412-16.
Roy H K, Kim Y L, Wali R K, Lui Y, Koetsier J, Kunte D P, Goldberg M J, Backman V, Cancer Epidemiol. Biomarkers Prev. 2005; 14(7). July 2005.
Roy H K, Lynch H T. Diagnosing Lynch syndrome: is the answer in the mouth? Gut 2003;52:1665-1667.
Roy H K, Olusola B F, Clemens D L, et al. AKT proto-oncogene overexpression is an early event during sporadic colon carcinogenesis. Carcinogenesis 2002;23:201-5.
Roy H K, Smyrk T C, Koetsier J, Victor T A, Wali R K. The transcriptional repressor SNAIL is overexpressed in human colorectal cancer. Dig Dis Sci. 2005 January;50(1):42-6.
Russello S V, Shore S K. SRC in human carcinogenesis. Front Biosci 2004;9: 139-44.
Sahu R K, Argov S, Bernshtain E, et al. Detection of abnormal proliferation in histologically “normal” colonic biopsies using FTIR-microspectroscopy. Scand J Gastroenterol 2004;39:557-66.
Sams J S, Lynch H T, Burt R W, Lanspa S J, Boland C R. Abnormalities of lectin histochemistry in familial polyposis coli and hereditary nonpolyposis colorectal cancer. Cancer 1990;66:502-8.
Sanders, M., “Molecular and cellular concepts in atherosclerosis,” Pharmacol. Ther., vol. 61, pp. 109-153, 1994.
Sarrio D, Perez-Mies B, Hardisson D, et al. Cytoplasmic localization of p120ctn and E-cadherin loss characterize lobular breast carcinoma from preinvasive to metastatic lesions. Oncogene 2004;23:3272-83.
Schmitt J M, Kumar G. Optical scattering properties of soft tissue: a discrete particle model. Appl Optics 1998;37:2788-2797.
Schwartz, S. M. and G. R. Campbell, “Replication of smooth muscle cells in vascular disease,” Circ. Res., vol. 598, pp. 427-444, 1986. J. Thyberg, K. Blomgren, J. Roy, P. K. Tran, and U. Hedin, “Phenotypic Modulation of Smooth Muscle Cells after Arterial Injury Is Associated with Changes in the Distribution of Laminin and Fibronectin,” J. Histochem. Cytochem., vol. 45, pp. 837-846, 1997.
Sharma R A, Dalgleish A G, Steward W P, et al. Angiogenesis and the immune response as targets for the prevention and treatment of colorectal cancer (review). Oncol Rep 2003; 10:1625-31.
Shpitz B, Gochberg S, Neufeld D, et al. Angiogenic switch in earliest stages of human colonic tumorigenesis. Anticancer Res 2003;23:5153-7.
Siddiq, M., C. Wu, B. Li. Journal of Applied Polymer Science 1996; 60: 1995.
Sirovich B E, Schwartz L M, Woloshin S. Screening men for prostate and colorectal cancer in the United States—does practice reflect the evidence? JAMA 2003;289:1414-1420.
Smits R, Ruiz P, Diaz-Cano S, et al. E-cadherin and adenomatous polyposis coli mutations are synergistic in intestinal tumor initiation in mice. Gastroenterology 2000; 119:1045-53.
Sokolov, K., R. A. Drezek, K. Gossage, and R. R. Richards-Kortum, “Reflectance spectroscopy with polarized light: Is it sensitive to cellular and nuclear morphology,” Opt. Exp., vol. 5, pp. 302-317, 1999.
Sperling, L. H, e. New York: John Wiley & Sons.1992.
Suggs, L. J, E. Y. Kao, L. L. Palombo, R. S. Krishnan, M. S. Widmer, A. G. Mikos, in Polymers for Tissue Engineering, (Eds: H. A. H. M. S. Shoichet). Utrecht: VSP. 1998 p. 99.
Summerton J, Weller D. Morpholino antisense oligomers: design, preparation, and properties. AntisenseNucleicAcid Drug Dev 1997;7:187-95.
Sun X F, Zhang H, Wu X C, et al. Microvascular corrosion casting of normal tissue, transitional mucosa and adenocarcinoma in the human colon. Acta Oncol 1992;31:37-40.
Szuromi, E, M. Berka, J. Borbaly. Macromolecules 2000; 33: 3993.
Takayama T, Katsuki S, Takahashi Y, Ohi M, Nojiri S, Sakamaki S, Kato J, Kogawa K, Miyake H, Niitsu Y. Aberrant crypt foci of the colon as precursors of adenoma and cancer. N Engl J Med 1998;339:1277-1284.
Teo N B, Shoker B S, Martin L, et al. Angiogenesis in pre-invasive cancers. Anticancer Res 2002;22:2061-72.
Torrance C J, Jackson P E, Montgomery E, et al. Combinatorial chemoprevention of intestinal neoplasia. Nat Med 2000;6:1024-8.
Tromberg B J, Shah N, Lanning R, Cerussi A, Espinoza J, Pham T, Svaasand L, Butler J. Non-invasive in vivo characterization of breast tumors using photon migration spectroscopy. Neoplasia 2000;2:26-40.
Van Dam J. Novel methods of enhanced endoscopic imaging. Gut 2003;52: 12-16.
van Wachem P B, Beugeling T, Feijen I, Biomaterials 1985;6:403-408.
Vega, J. R., L. M. Gugliotta, V. D. G. Gonzalez, G. R. Meira. Journal of Colloid and Interface Science 2003; 261: 74.
Vucenik I, Gotovac J, Druzijanic N, Shamsuddin A M. Usefulness of galactose oxidase-Schiff test in rectal mucus for screening of colorectal malignancy. Anticancer Res 2001;21:1247-1255.
Wali R K, Roy H K, Kim Y, et al. Increased microvascular blood content is an early event in colon carcinogenesis. Gut 2005;54:654-60.
Wali R K, Roy H K, Kim Y, et al. Increased mucosal blood flow is an early marker of colon carcinogenesis. Gastroenterology 2003; 124(suppl):A4.
Waliszewski P, Molski M, Konarski J. On the holistic approach in cellular and cancer biology: nonlinearity, complexity, and quasideterminism of the dynamic cellular network. J Surg Oncol 1998; 68:70-78.
Wallace M B, Perelman L T, Backman V, Crawford J M, Fitzmaurice M, Seiler M, Badizadegan K, Shields S J, Itzkan I, Dasari R R, Van Dam J, Feld M S. Endoscopic detection of dysplasia in patients with Barrett's esophagus using light-scattering spectroscopy. Gastroenterology 2000; 11 9:677-682.
Wang Y D, Ameer G A, Sheppard B I, Langer R. Nature Biotechnology, 2002;20:602-606.
Watson P, Ashwathnarayan R, Lynch H T, Roy H K. Tobacco use and increased colorectal cancer risk in patients with hereditary nonpolyposis colorectal cancer (Lynch syndrome). Arch Intern Med 2004;164:2429-31.
Wax A, Yang C H, Backman V, Badizadegan K, Boone C W, Dasari R R, Feld M S. Cellular organization and substructure measured using angle-resolved low-coherence interferometry. Biophys J 2002;82:2256-2264.
Wax A, Yang C H, Muller M G, Nines R, Boone C W, Steele V E, Stoner G D, Dasari R R, Feld M S. In situ detection of neoplastic transformation and chemopreventive effects in rat esophagus epithelium using angle-resolved low-coherence interferometry. Cancer Res 2003;63:3556-3559.
Weyn B, Jacob W, da Silva V D, Montironi R, Hamilton P W, Thompson D, Bartels H G, Van Daele A, Dillon K, Bartels P H. Data representation and reduction for chromatin texture in nuclei from premalignant prostatic, esophageal, and colonic lesions. Cytometry 2000;41:133-138.
Willett C G, Boucher Y, di Tomaso E, et al. Direct evidence that the VEGF specific antibody bevacizumab has antivascular effects in human rectal cancer. Nat Med 2004; 10:145-7.
Winawer S, Fletcher R, Rex D, Bond J, Burt R, Ferrucci J, Ganiats T, Levin T, Woolf S, Johnson D, Kirk L, Litin S, Simmang C. Colorectal cancer screening and surveillance: clinical guidelines and rationale-update based on new evidence. Gastroenterology 2003;124:544-560.
Xu M H, Deng C S, Zhu Y Q, et al. Role of inducible nitric oxide synthase expression in aberrant crypt foci-adenoma-carcinoma sequence. World J Gastroenterol 2003;9:1246-50.
Xue L, Greisler H P I. Vas. Surg.2003;37:472-480.
Yamada Y, Mori H. Pre-cancerous lesions for colorectal cancers in rodents: a new concept. Carcinogenesis 2003;24:1015-1019.
Yang J, Bei J Z, Wang S G. Biomaterials, 2002;23:2607-2614.
Yang J, Shi G X, Bei J Z, Wang S G, Cao Y L, Shang Q X, Yang G H, Wang W J. J. Biomed. Mater. Res. 2002, 62(3):438.446.
Yodh A G, Chance B. Spectroscopy and imaging with diffusing light. Physics Today 1995; 48:34-40.
Yokoyama K, Kamata N, Hayashi E, et al. Reverse correlation of E-cadherin and snail expression in oral squamous cell carcinoma cells in vitro. Oral Oncol 2001;37:65-71.
Yoshikawa R, Utsunomiya J. Cell proliferation kinetics are abnormal in transitional mucosa adjacent to colorectal carcinoma. Br J Surg 1996;83:36-39.
Zack D L, DiBaise J K, Quigley E M M, Roy H K. Colorectal cancer screening compliance by medicine residents: perceived and actual. Am J Gastroenterol 2001;96:3004-3008.
Zanten, J. H. v., H. G. Monbouquette. Journal of Colloid and Interface Science 1994; 165:
Ziegler T, Nerem R M. I. Cell. Biochem. 1994;56:204-209.
Zonios G, Perelman L T, Backman V M, Manoharan R, Fitzmaurice M, Van Dam J, Feld M S. Diffuse reflectance spectroscopy of human adenomatous colon polyps in vivo. Appl Optics 1999;38: 6628-6637.

1. A method of examining a sample, comprising:
measuring, as function of wavelength of light elastically scattered from the sample, at least 2 properties, selected from the group consisting of scattering angle theta of the light, scattering angle phi of the light, and polarization of the light;
wherein the scattering angle theta is an angle between backward direction and direction of propagation of the light, and
the scattering angle phi is an angle between incident light polarization and projection of direction of the light propagation onto a plane in which incident electric field oscillates.

2. The method of claim 1, comprising measuring, as function of wavelength of light elastically scattered from the sample, the scattering angle theta of the light, the scattering angle phi of the light, and the polarization of the light.

3. The method of claim 2, wherein the measuring comprising measuring the scattering angle theta of the light, the scattering angle phi of the light, and the polarization of the light for at least two different values of wavelength, scattering angle theta, or scattering angle phi.

4. The method of claim 3, wherein the at least two different values is at least 4 different values.

5. The method of claim 3, wherein the at least two different values is at least 12 different values.

6. The method of claim 3, wherein the measuring comprises measuring the scattering angle theta for at least two different values of scattering angle theta, and the two different values have a difference of 1 to 10 degrees.

7. The method of claim 3, wherein the wavelength, the scattering angle theta, the scattering angle phi, and the polarization are measured simultaneously for each scattering angle theta.

8. The method of claim 1, wherein the wavelength of the light scattered comprises light having a wavelength from infrared to ultraviolet.

9. The method of claim 1, wherein the wavelength of the light scattered comprises visible light.

10. The method of claim 1, wherein the sample is a biological sample.

11. The method of claim 10, wherein a living organism comprises the sample.

12. The method of claim 11, wherein the organism is a human patient.

13. The method of claim 1, wherein the sample comprises a translucent polymer.

14. A method of screening a patient for cancer, comprising:
examining a sample by the method of claim 2, wherein the sample is from the patient.

15. The method of claim 14, wherein the sample is measured in vivo.

16. The method of claim 15, wherein the measuring comprising measuring the scattering angle theta of the light, the scattering angle phi of the light, and the polarization of the light for at least two different values of wavelength, scattering angle theta, and/or scattering angle phi.

17. The method of claim 16, wherein the at least two different values is at least 4 different values.

18. The method of claim 16, wherein the at least two different values is at least 12 different values.

19-35. (canceled)

36. A multi-dimensional elastic light scattering instrument, comprising:
(i) a light delivery system, for delivering a collimated linearly polarized beam of light to a sample,
(ii) a light collection system, for collecting light from the light delivery system scattered from the sample, and
(iii) optionally, a calibration system,
wherein the instrument measures, as function of wavelength of light elastically scattered from the sample, scattering angle theta of the light, scattering angle phi of the light, and polarization of the light,
the scattering angle theta is an angle between backward direction and direction of propagation of the light, and
the scattering angle phi is an angle between incident light polarization and projection of direction of the light propagation onto a plane in which incident electric field oscillates.

37-40. (canceled)

41. A multi-dimensional elastic light scattering probe, comprising:
(a) a first optical fiber,
(b) a first set of at least one optical fiber, and
(c) a second set of at least one optical fiber,
wherein the first optical fiber, the first set, and the second set, all have an end optically coupled to an end of the probe, and
the probe has an outer diameter of at most 1.5 mm.

42-47. (canceled)
